(19) United States
(12) Patent Application Publication
(10) Pub. No.: US 2025/0239372 A1
(43) Pub. Date: Jul. 24, 2025

(54) T Cell Quantitative Systems Pharmacology Model

(71) Applicant: Genentech, Inc., South San Francisco, CA (US)

(72) Inventors: Iraj Hosseini, San Carlos, CA (US); Louis Russel Joslyn, San Francisco, CA (US); Saroja Ramanujan, San Mateo, CA (US)

(21) Appl. No.: 19/177,222

(22) Filed: Apr. 11, 2025

Related U.S. Application Data
(63) Continuation of application No. PCT/US2023/076629, filed on Oct. 11, 2023.
(60) Provisional application No. 63/415,649, filed on Oct. 12, 2022.

(51) Int. Cl. G16H 50/50; G16B 5/30; G16H 10/60; G16H 20/10

(52) U.S. Cl. CPC G16H 50/50; G16B 5/30; G16H 10/60; G16H 20/10

(57) ABSTRACT

A method may include determining, by at least one data processor and based on a set of cellular kinetics parameters corresponding to a plurality of T cell phenotypes and trafficking rates of the plurality of T cell phenotypes between a peripheral tissue and lymph node compartment (i.e., healthy tissue compartment), a blood compartment, a tumor draining lymph node compartment, and a tumor compartment, a distribution of the plurality of T cell phenotypes over time. The plurality of T cell phenotypes may include a stem-like memory T cell, a central memory T cell, an effector memory T cell, an effector T cell, and an endogenous T cell. Related methods and articles of manufacture are also disclosed.

### PRIORITY

This application is a continuation under 35 U.S.C. § 365 (c) of International Patent Application No. PCT/US2023/076629, filed 11 Oct. 2023, which claims the benefit under 35 U.S.C. § 119 (e) of U.S. Provisional Patent Application No. 63/415,649, filed 12 Oct. 2022, each of which is incorporated herein by reference.

### TECHNICAL FIELD

The present disclosure generally relates to quantitative systems pharmacology models, and more specifically to T cell quantitative systems pharmacology models.

### BACKGROUND

T cell receptor (TCR)-engineered T cell therapy is an emerging cancer treatment strategy that has shown evidence of anti-tumor activity in both solid tumors and hematologic cancer. Despite initial promise, there remain many challenges in understanding and characterizing the unique cellular kinetics, including trafficking, proliferation, apoptosis, and persistence of TCR-engineered T cells, among other T cells and T cell therapies, following infusion into the patient. As TCR-engineered T cell therapies are comprised of live and phenotypically diverse T cells, the pharmacological properties of these therapies differ from molecular therapeutics, and monitoring and predicting the pharmacokinetic and resulting pharmacodynamics of these therapies poses distinct challenges. Further, it is not yet known how the cellular phenotype and composition of the infusion product might affect the cellular pharmacokinetics and subsequent efficacy and safety.

### SUMMARY OF PARTICULAR EMBODIMENTS

Methods, systems, and articles of manufacture, including computer program products, are provided for T cell quantitative systems pharmacology models. In one aspect, there is provided a method. The method may comprise, generating a model, wherein the model simulates a distribution of each of a plurality of T cell phenotypes in a plurality of physiological compartments over time after delivery a T cell target (TCT) product to a patient; generating a plurality of digital twins by the model, wherein each of the plurality of digital twins represents a distribution of the plurality of T cell phenotypes in the plurality of physiological compartments over time associated with a corresponding patient, wherein the corresponding patient has a set of patient characteristics; receiving a set of characteristics associated with a target patient; matching the target patient characteristics with the patient characteristics associated with digital twins to select a subset of digital twins that resemble the target patient from the plurality of digital twins; predicting target patient responses to a plurality of hypothetical deliveries of the TCT product with different dose and composition of T cell phonotypes using the selected subset of digital twins for the target patient, and generating a treatment plan for the target patient based on the predicted responses provided by the subset of digital twins, wherein the treatment plan is predicted to provide a T cell persistence level above a predetermined threshold over time in at least one of the plurality of physiological compartments of the target patient, wherein the treatment plan comprises dose and composition of the plurality of T cell phonotypes of the treatment.

In some variations, the method further comprises identifying sources of variability across patients by visualizing a parameter space for a second subset of the plurality of digital twins; and modifying the model to account for the sources of variability across patients.

In some variations, the method further comprises generating a plurality of simulations across different T cell phonotypes composition and dose level by the model; visualizing a distribution of each of the plurality of T cell phenotypes over time associated with the plurality of simulations, wherein the visualization is indicative of an impact of T cell phonotypes composition on T cell persistence level over time.

In some variations, the set of patient characteristics comprises patient biometrics, patient medical history, and baseline biomarker data.

In some variations, each of the plurality of digital twins further represents a response to a dose of a composition of the TCT product within the plurality of compartments.

In some variations, each of the plurality of digital twins comprises a set of cellular kinetics parameters including at least one of a quantity, a proliferation rate, a trafficking rate, an apoptosis rate, and a differentiation rate of the plurality of T cell phenotypes within at least one of the plurality of physiological compartments of the corresponding patient over a period of time.

In some variations, the composition of TCT product comprises an initial quantity of each of the plurality of T cell phenotypes.

In some variations, the plurality of T cell phenotypes comprises at least two of a stem-like memory T cell, a central memory T cell, an effector memory T cell, an effector T cell, and an endogenous T cell.

In some variations, the plurality of physiological compartments includes a peripheral tissue and lymph node compartment, a blood compartment, a tumor draining lymph node compartment, and a tumor compartment.

Methods, systems, and articles of manufacture, including computer program products, are provided for T cell quantitative systems pharmacology models. In one aspect, there is provided a method. The method may include determining, by at least one data processor, a set of cellular kinetics parameters corresponding to a plurality of T cell phenotypes within a peripheral tissue and lymph node compartment (i.e., healthy tissue compartment), the blood compartment, the tumor draining lymph node compartment, and the tumor compartment of a patient after delivery of a T cell target (TCT) product. The plurality of T cell phenotypes includes a stem-like memory T cell, a central memory T cell, an effector memory T cell, an effector T cell, and an endogenous T cell. The method may include determining, by the at least one data processor, a first trafficking rate of the plurality of T cell phenotypes between the peripheral tissue and lymph node compartment and a blood compartment of the patient. The method may include determining, by the at least one data processor, the set of cellular kinetics parameters corresponding to the plurality of T cell phenotypes within the blood compartment. The method may include determining, by the at least one data processor, a second trafficking rate of the plurality of T cell phenotypes between the blood compartment and a tumor draining lymph node compartment of the patient. The method may include determining, by the at least one data processor, the set of cellular kinetics parameters corresponding to the plurality of T cell phenotypes within the tumor draining lymph node compartment. The method may include determining, by the at least one data processor, a third trafficking rate of the effector memory T cell and the effector T cell from the blood compartment to a tumor compartment of the patient. The method may include determining, by the at least one data processor, the set of cellular kinetics parameters corresponding to the effector memory T cell and the effector T cell within the tumor compartment. The method may include determining, by the at least one data processor and based on the set of cellular kinetics parameters, the first trafficking rate, the second trafficking rate, and the third trafficking rate, a distribution of each of the plurality of T cell phenotypes in the peripheral tissue and lymph node compartment (i.e., healthy tissue compartment), the blood compartment, the tumor draining lymph node compartment, and the tumor compartment over time.

In some variations, one or more of the features disclosed herein including the following features can optionally be included in any feasible combination. In some variations, the method includes determining, by the at least one data processor, a differentiation rate parameter corresponding to a differentiation rate of the effector memory T cell into the effector T cell within the tumor compartment. The determining the distribution is further based on the differentiation rate parameter.

In some variations, the method includes determining, by the at least one data processor, a differentiation rate parameter corresponding to a differentiation of the stem-like memory T cell into the central memory T cell, the central memory T cell into the effector memory T cell, and the effector memory T cell into the effector T cell within the tumor draining lymph node compartment. The determining the distribution is further based on the differentiation rate parameter.

In some variations, the method includes determining a T cell therapy for treating a tumor, wherein the T cell therapy includes a dose of a composition of the plurality of T cell phenotypes of the TCT product.

In some variations, the T cell therapy is at least one of a T cell receptor (TCR)-engineered T cell therapy, an autologous T cell therapy, an allogeneic T cell therapy, an iPSC-derived T cell therapy, and a CAR T cell therapy.

In some variations, the TCT product includes a dose of a composition of the plurality of T cell phenotypes.

In some variations, the tumor compartment includes a tumor site of a tumor.

In some variations, the distribution includes: a first distribution corresponding to a dose of a composition of the TCT product administered to a first patient and a second distribution corresponding to a dose of a composition of the TCT product administered to a second patient. The method includes: determining, by the at least one data processor and based at least on the first distribution and the second distribution, a response of a third patient to a T cell therapy including a dose of a composition of the TCT product.

In some variations, the method includes determining, based at least on the response of the third patient to the T cell therapy, a treatment plan for the third patient.

In some variations, the dose of the composition of the TCT product administered to the first patient is administered to the first patient after administration of a lymphodepletion regimen to the first patient. The dose of the composition of the TCT product administered to the second patient is administered to the second patient after administration of another lymphodepletion regimen.

In some variations, the set of cellular kinetics parameters includes at least one of a quantity, a proliferation rate, an apoptosis rate, and a differentiation rate of the plurality of T cell phenotypes.

In one aspect, a method includes determining, by at least one data processor, a first patient profile representing a first response to a first dose of a first composition of a T cell target (TCT) product within a plurality of physiological compartments of a first patient. The method includes determining, by the at least one data processor, a second patient profile representing a second response to a second dose of a second composition of the TCT product within the plurality of physiological compartments of a second patient. The method includes generating, by the at least one data processor and based at least on the first patient profile and the second patient profile, an output indicating a pharmacokinetic-pharmacodynamic (PKPD) relationship for the TCT product. The method includes determining, by the at least one data processor and based at least on the output, a response of a third patient to a T cell therapy including a third dose of a third composition of the TCT product. The method includes determining, based at least on the response of the third patient to the T cell therapy including the third dose of the third composition of the TCT product, a treatment plan for the third patient.

In some variations, the first patient profile includes a first set of cellular kinetics parameters including at least one of a first quantity, a first proliferation rate, a first trafficking rate, a first apoptosis rate, and a first differentiation rate of a plurality of T cell phenotypes within at least one of the plurality of physiological compartments of the first patient over a period of time. The second patient profile includes a second set of cellular kinetics parameters including at least one of a second quantity, a second proliferation rate, a second trafficking rate, a second apoptosis rate, and a second differentiation rate of the plurality of T cell phenotypes within at least one of the plurality of physiological compartments of the second patient over the period of time.

In some variations, the first composition includes a first quantity of each of a plurality of T cell phenotypes. The second composition includes a second quantity of each of the plurality of T cell phenotypes.

In some variations, the first patient profile and the second patient profile each include responses corresponding to each of the plurality of T cell phenotypes.

In some variations, the plurality of T cell phenotypes includes at least two of a stem-like memory T cell, a central memory T cell, an effector memory T cell, an effector T cell, and an endogenous T cell.

In some variations, the plurality of physiological compartments includes a peripheral tissue and lymph node compartment, a blood compartment, a tumor draining lymph node compartment, and a tumor compartment.

In some variations, the T cell therapy is at least one of a T cell receptor (TCR)-engineered T cell therapy, an autologous T cell therapy, an allogeneic T cell therapy, an iPSC-derived T cell therapy, and a CAR T cell therapy.

In some variations, a linear increase from the first dose to the second dose results in a nonlinear change between the first response and the second response.

In some variations, the first composition is different from the second composition. The first dose is different from the second dose.

In some variations, determining the response in the third patient to the T cell therapy includes: simulating, based at least on the output, a plurality of responses to a plurality of doses of a plurality of compositions of the TCT product in a plurality of simulated patients. The response in the third patient is one of the plurality of simulated responses.

In some variations, the first patient profile is determined after administration of a first lymphodepletion regimen to the first patient. The second patient profile is determined after administration of a second lymphodepletion regimen to the second patient.

In some variations, the response in the third patient is determined by at least varying at least one of the first dose, the first composition, the second dose, and the second composition.

In some variations, the simulating the plurality of responses is based on application of a lymphodepletion regimen to the plurality of simulated patients.

In one aspect, there is provided a system. The system may include at least one processor and at least one memory. The at least one memory may store instructions that result in operations when executed by the at least one processor. The operations may include: determining a set of cellular kinetics parameters corresponding to a plurality of T cell phenotypes within a peripheral tissue and lymph node compartment (i.e., healthy tissue compartment) of a patient after delivery of a T cell target (TCT) product. The plurality of T cell phenotypes includes a stem-like memory T cell, a central memory T cell, an effector memory T cell, an effector T cell, and an endogenous T cell. The operations may include determining a first trafficking rate of the plurality of T cell phenotypes between the peripheral tissue and lymph node compartment (i.e., healthy tissue compartment) and a blood compartment of the patient. The operations may include determining the set of cellular kinetics parameters corresponding to the plurality of T cell phenotypes within the blood compartment. The operations may include determining a second trafficking rate of the plurality of T cell phenotypes between the blood compartment and a tumor draining lymph node compartment of the patient. The operations may include determining the set of cellular kinetics parameters corresponding to the plurality of T cell phenotypes within the tumor draining lymph node compartment. The operations may include determining a third trafficking rate of the effector memory T cell and the effector T cell from the blood compartment to a tumor compartment of the patient. The operations may include determining the set of cellular kinetics parameters corresponding to the effector memory T cell and the effector T cell within the tumor compartment. The operations may include determining, by the at least one data processor and based on the set of cellular kinetics parameters, the first trafficking rate, the second trafficking rate, and the third trafficking rate, a distribution of each of the plurality of T cell phenotypes in the peripheral tissue and lymph node compartment, the blood compartment, the tumor draining lymph node compartment, and the tumor compartment over time.

In another aspect, there is provided a system. The system may include at least one processor and at least one memory. The at least one memory may store instructions that result in operations when executed by the at least one processor. The operations may include determining, by at least one data processor, a first patient profile representing a first response to a first dose of a first composition of a T cell target (TCT) product within a plurality of physiological compartments of a first patient. The operations may include determining, by the at least one data processor, a second patient profile representing a second response to a second dose of a second composition of the TCT product within the plurality of physiological compartments of a second patient. The operations may include generating, by the at least one data processor and based at least on the first patient profile and the second patient profile, an output indicating a pharmacokinetic-pharmacodynamic (PKPD) relationship for the TCT product. The operations may include determining, by the at least one data processor and based at least on the output, a response of a third patient to a T cell therapy including a third dose of a third composition of the TCT product. The operations may include determining, based at least on the response of the third patient to the T cell therapy including the third dose of the third composition of the TCT product, a treatment plan for the third patient.

In another aspect, there is provided a computer program product that includes a non-transitory computer readable storage medium. The non-transitory computer-readable storage medium may include program code that causes operations when executed by at least one processor. The operations may include: determining a set of cellular kinetics parameters corresponding to a plurality of T cell phenotypes within a peripheral tissue and lymph node compartment (i.e., healthy tissue compartment) of a patient after delivery of a T cell target (TCT) product. The plurality of T cell phenotypes includes a stem-like memory T cell, a central memory T cell, an effector memory T cell, an effector T cell, and an endogenous T cell. The operations may include determining a first trafficking rate of the plurality of T cell phenotypes between the peripheral tissue and lymph node compartment (i.e., healthy tissue compartment) and a blood compartment of the patient. The operations may include determining the set of cellular kinetics parameters corresponding to the plurality of T cell phenotypes within the blood compartment. The operations may include determining a second trafficking rate of the plurality of T cell phenotypes between the blood compartment and a tumor draining lymph node compartment of the patient. The operations may include determining the set of cellular kinetics parameters corresponding to the plurality of T cell phenotypes within the tumor draining lymph node compartment. The operations may include determining a third trafficking rate of the effector memory T cell and the effector T cell from the blood compartment to a tumor compartment of the patient. The operations may include determining the set of cellular kinetics parameters corresponding to the effector memory T cell and the effector T cell within the tumor compartment. The operations may include determining, by the at least one data processor and based on the set of cellular kinetics parameters, the first trafficking rate, the second trafficking rate, and the third trafficking rate, a distribution of each of the plurality of T cell phenotypes in the peripheral tissue and lymph node compartment, the blood compartment, the tumor draining lymph node compartment, and the tumor compartment over time.

In another aspect, there is provided a computer program product that includes a non-transitory computer readable storage medium. The non-transitory computer-readable storage medium may include program code that causes operations when executed by at least one processor. The operations may include determining, by at least one data processor, a first patient profile representing a first response to a first dose of a first composition of a T cell target (TCT) product within a plurality of physiological compartments of a first patient. The operations may include determining, by the at least one data processor, a second patient profile representing a second response to a second dose of a second composition of the TCT product within the plurality of physiological compartments of a second patient. The operations may include generating, by the at least one data processor and based at least on the first patient profile and the second patient profile, an output indicating a pharmacokinetic-pharmacodynamic (PKPD) relationship for the TCT product. The operations may include determining, by the at least one data processor and based at least on the output, a response of a third patient to a T cell therapy including a third dose of a third composition of the TCT product. The operations may include determining, based at least on the response of the third patient to the T cell therapy including the third dose of the third composition of the TCT product, a treatment plan for the third patient.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to T cell quantitative systems pharmacology models, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts an exemplary T cell phenotype analysis system, consistent with implementations of the current subject matter.

FIG. 2 depicts an exemplary architecture of a QSP model, consistent with implementations of the current subject matter.

FIG. 3 depicts an exemplary graph illustrating cell counts over time, consistent with implementations of the current subject matter.

FIG. 4 depicts an exemplary graph illustrating a set of cellular kinetics parameters for a T cell phenotype, for example, a single simulation that shows the model capturing known dynamics of T cell therapies across time, consistent with implementations of the current subject matter.

FIG. 5 depicts an exemplary comparison of cellular pharmacokinetics in blood, consistent with implementations of the current subject matter.

FIGS. 6A and 6B depict exemplary comparison of cellular pharmacokinetics in blood across various dose groups and patients, consistent with implementations of the current subject matter.

FIG. 7 depicts an exemplary graph showing changes in cellular pharmacokinetics based on phenotypic makeup of a T cell product and dose level, consistent with implementations of the current subject matter.

FIG. 8 depicts an exemplary graph showing changes in cellular pharmacokinetics based on phenotypic makeup of a T cell product and dose level, consistent with implementations of the current subject matter.

FIG. 9 depicts a flowchart illustrating an example of a process for generating a distribution of T cell phenotypes, consistent with implementations of the current subject matter.

FIG. 10 depicts a flowchart illustrating an example of a process for determining a patient response to a T cell therapy, consistent with implementations of the current subject matter.

FIG. 11 depicts a block diagram illustrating an example of a computing system, consistent with implementations of the current subject matter.

FIG. 12 depicts exemplary parameter space ridgeline plots and principal component analysis of digital twins reveal sources of variability between patients and across dose groups (A-O).

FIG. 13 depicts exemplary biological variability (patient-specific) impacts cellular kinetics of TCR-engineered T cell therapies leading to persister or non-persister outcomes.

FIG. 14 depicts exemplary graphs showing dose composition impacts cellular kinetics of TCR-engineered T cells.

FIG. 15 depicts exemplary predictive simulations of digital twins demonstrate alignment with observed profiles in patients with available clinical data.

FIG. 16 depicts a flowchart illustrating an example of a process for generating personalized treatment plans using the model and the digital twins, consistent with implementations of the current subject matter.

When practical, like labels are used to refer to same or similar items in the drawings.

### DESCRIPTION OF EXAMPLE EMBODIMENTS

As noted, there are many challenges in understanding and characterizing the unique cellular kinetics of T cell therapies, such as when the T cell therapies are unconventional in that the therapies include live and phenotypically diverse T cells, the pharmacological properties of these therapies differ from molecular therapeutics, and monitoring and predicting the pharmacokinetic and resulting pharmacodynamics of these therapies pose distinct challenges. Conventional pharmacokinetic-pharmacodynamic (PKPD) approaches may not be sufficient in such instances. Further, it is not yet known how the cellular phenotype and composition of the infusion product might affect the cellular pharmacokinetics (e.g., cellular kinetics) and subsequent efficacy and safety. For example, there is currently a lack of adequate animal models and preclinical data for understanding the translation of T cell therapies, such as engineered TCR T cell therapies. Moreover, the cellular kinetics of such T cell therapies can be significantly influenced by an administered dose, variability in composition, and variability in patient characteristics and/or make-up.

To address these challenges, the architecture of the quantitative systems pharmacology (QSP) model provided herein, consistent with implementations of the current subject matter, accounts for variability in the dose and/or composition of the T cell therapies, and variability in patients, to accurately predict distributions of a plurality of T cell phenotypes in various physiological compartments and/or patient responses, over time. For example, the QSP model described herein may track T cell pharmacokinetics of different subsets of TCR-engineered T cells, such as stem-like memory T cells (Tscm), central memory T cells (Tcm), effector memory T cells (Tem), effector T cells (Teff), and/or endogenous T cells across physiological compartments, including a tissue and lymph node compartment, a blood compartment, a tumor draining lymph node compartment, and a tumor compartment. Each subpopulation undergoes homeostatic proliferation, antigen-driven proliferation and differentiation, apoptosis, margination and trafficking between and/or within the physiological compartments. In addition, as described herein, the architecture of the QSP model captures the effector memory T cell and effector T cell populations infiltrating the tumor compartment, where the effector T cells can kill tumor cells of the tumor within the tumor compartment. The architecture of the QSP model described herein may also capture the potential competition between immune cells following infusion with TCR-engineered T cells, by incorporating lymphocyte depletion conditioning therapy and subsequent proliferation of endogenous T cells (Tendo). As described herein, the QSP model was calibrated to phase I clinical trial data of TCR-engineered T cells targeting E7 in epithelial cancer patients. The QSP model described herein may be adaptable to other cancer antigens and tumor types. Accordingly, the QSP model described herein accurately predicts the impact of varying dose and T cell phenotype ratios (e.g., compositions) of various T cell therapies.

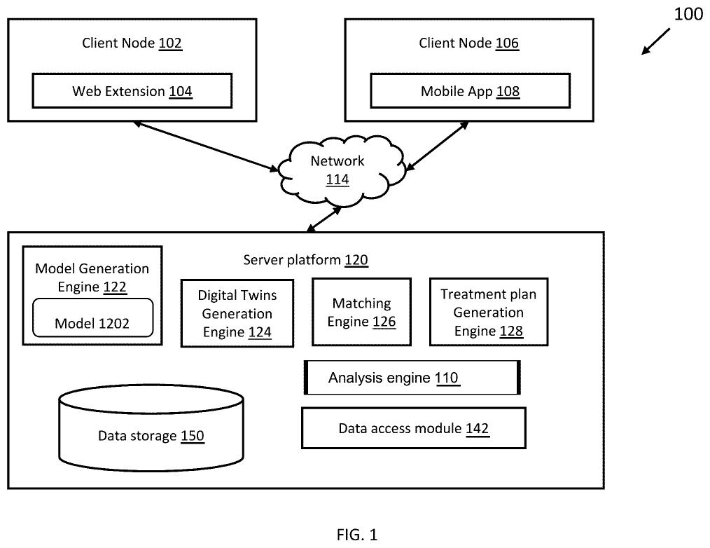

FIG. 1 is a block diagram depicting an example system 100 comprising a client-server architecture and network configured to perform the various methods described herein. A platform (e.g., machines and software, possibly interoperating via a series of network connections, protocols, application-level interfaces, and so on), in the form of a server platform 120, provides server-side functionality via a communication network 114 (e.g., the Internet or other types of wide-area networks (WANs), such as wireless networks or private networks with additional security appropriate to tasks performed by a user) to one or more client nodes 102, and/or 106.

A client node (e.g., client node 102 and/or client node 106) may be, for example, a user device (e.g., mobile electronic device, stationary electronic device, etc.). A client node may be associated with, and/or be accessible to, a user. In another example, a client node may be a computing device (e.g., server) accessible to, and/or associated with, an individual or entity. A node may comprise a network module (e.g., network adaptor) configured to transmit and/or receive data. Via the nodes in the computer network, multiple users and/or servers may communicate and exchange data. In some embodiments, the client node may facilitate transmission of patient data to the platform 120 for further processing.

As shown in FIG. 1, a client node 102 hosting a web extension 104, thus allowing a user to access functions provided by the server platform 120, for example, receiving a visualization of one or more of treatment plans from the server platform 120. The web extension 104 may be compatible with any web browser application used by a user of the client node. Further, FIG. 1 illustrates, for example, another client node 106 hosting a mobile application 108, thus allowing a user to access functions provide by the server platform 120, for example, receiving a visualization of one or more of treatment plans from the server platform 120. Delivery of the visualization may be through a wired or wireless mode of communication.

In at least some examples, the server platform 120 may be one or more computing devices or systems, storage devices, and other components that include, or facilitate the operation of, various execution modules depicted in FIG. 1. These modules may include, for example, a model generation engine 122, a digital twins generation engine 124, a matching engine 126, a treatment plan generation engine 128, a data access module 142, an analysis engine 110, and a data storage 150. Each of these modules is described in greater detail below.

The model generation engine 122 may generate a model that may capture the biomedical mechanism of the TCT product after injection into a patient's body. In some embodiments, the model generation engine 122 may collaborate with the analysis engine 110 to generate the model. The model generation operations performed by the analysis engine 110 and the model generation engine 122 are described in further details herein elsewhere. The digital twins generation engine 124 may facilitate the generation of a collection of digital twins. In some embodiments, the digital twins generation engine 124 may collaborate with the analysis engine 110 to generate the collection of digital twins. In some embodiments, the digital twins may be designed in a manner where each patient from the clinical trial is matched to a subset of specific digital twins by, for example, the matching engine 126. For example, using the model 1202, the digital twins generation engine 124 may generate a few hundreds, a few thousands or more digitals twins. In some embodiments, each of the plurality of digital twins may represent a distribution of the plurality of T cell phenotypes in the plurality of physiological compartments over time associated with a corresponding patient, wherein the corresponding patient may be associated with a set of patient characteristics. The matching engine 126 may receive a set of target patient characteristics from one or more of the client nodes 102 or 106, and may match the target patient characteristics with the patient characteristics associated with digital twins to select a subset of digital twins that resemble the target patient from the plurality of digital twins. The treatment plan generation engine 128 may collaborate with other engines/modules of the server platform 120 to generate treatment plans for one or more target patients. The treatment plan generation operation performed by the treatment plan generation engine 128 is described in further details herein elsewhere.

The data access modules 142 may facilitate access to data storage 150 of the server platform 120 by any of the remaining modules/engines 110, 122, 124, 126, and 128 of the server platform 120. In one example, one or more of the data access modules 142 may be database access modules, or may be any kind of data access module capable of storing data to, and/or retrieving data from, the data storage 150 according to the needs of the particular modules 110, 122, 124, 126, and 128 employing the data access modules 142 to access the data storage 150. Examples of the data storage 150 include, but are not limited to, one or more data storage components, such as magnetic disk drives, optical disk drives, solid state disk (SSD) drives, and other forms of nonvolatile and volatile memory components.

The data storage 150 may store input clinical data and/or one or more determinations/models/digital twins made and/or generated by the remaining modules/engines 110, 122, 124, 126, and 128 of the server platform 120. The data storage 150 may comprise a graph database, a time series database, a relational database, or a combination of these, to efficiently manage and organize a multitude of data related to T cell therapy. This data encompasses various aspects of patient responses to the TCT product, including a comprehensive collection of patient profiles/digital twins representing the distribution of T cell phenotypes within different physiological compartments over time.

In some embodiments, a graph database may be utilized because of its inherent capability to efficiently model and manage intricate relationships within complex datasets. For example, as discussed herein elsewhere, T cell therapy research may involve a web of intricate relationships among various data entities, including different T cell phenotypes, distribution and/or concentration of the different T cell phenotypes in different physiological compartments, and cellular kinetics parameters. A graph database may be able to represent this interconnected data structures. In some embodiments, graph databases may be utilized by employing nodes and edges to represent entities and their relationships, and therefore modeling the interconnectedness of different components in the T cell therapy domain. For example, nodes can represent T cell phenotypes, compartments, and parameters, while edges depict the relationships and interactions between them. Additionally or alternatively, graph databases may offer flexibility in querying and traversing relationships. For example, it may visualize how different T cell phenotypes influence each other, how different T cell phenotypes move between compartments, and how changes in cellular kinetics parameters affect distributions over time. This may provide insights into the dynamics of T cell response.

In some embodiments, a time series database may be useful for handling data that evolves over time, for example, the distribution of a plurality of T cell phenotypes in a plurality of physiological compartments over time. A time series database may capture the temporal and dynamic aspects of patient responses, enabling visibility to changes in T cell phenotypes within physiological compartments as therapy progresses. For example, time series databases may be utilized to store and query time-stamped data points, which may facilitate tracking of cellular kinetics parameters over time. In some embodiments, a relational database may be utilized to store structured clinical data and metadata associated with T cell therapy, facilitating the linking of patient profiles to specific treatment regimens, laboratory results, and patient demographics. A relational database may advance data integrity and enable direct retrieval of specific pieces of information.

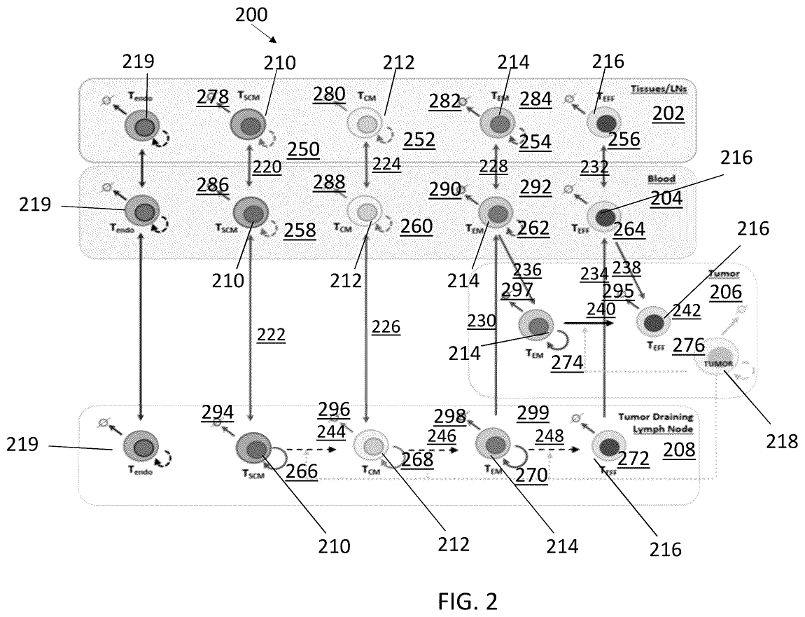

FIG. 2 schematically depicts an example architecture 200 of the QSP model 1202, consistent with implementations of the current subject matter. As noted, the QSP model 1202 describes in vivo dynamics of lymphocyte (e.g., T cell) proliferation following treatment with a T cell target (TCT) product, consistent with embodiments of the current subject matter. In other words, the architecture 200 of the QSP model 1202 may represent the pharmacokinetic-pharmacodynamic relationship of the TCT product. The TCT product may additionally and/or alternatively increase anti-tumor activity in both solid tumors and hematologic cancers. For example, the TCT product may be delivered to a patient as part of a T cell therapy for treating tumors, cancers, and/or the like. The T cell therapy may be at least one of a T cell receptor (TCR)-engineered T cell therapy, an autologous T cell therapy, an allogeneic T cell therapy, an induced pluripotent stem cell (“iPSC”) derived T cell therapy, and a chimeric antigen receptor (“CAR”) T cell therapy.

The TCT product may be composed of a plurality of T cell phenotypes that proliferate within various physiological compartments of a patient, as described herein. For example, the TCT product may include a dose of a composition of a plurality of T cell phenotypes. Referring to FIG. 2, the plurality of T cell phenotypes includes a stem-like memory T cell (Tscm) 210, a central memory T cell (Tcm) 212, an effector memory T cell (Tem) 214, an effector T cell (Teff) 216, and/or an endogenous T cell 219. The particular T cell phenotypes of the plurality of T cell phenotypes included in the architecture 200 were selected to improve the accuracy of predictions generated based on the QSP model 1202, to more accurately track T cell phenotype behavior within and between a plurality of physiological compartments, and to more accurately determine distributions of the T cell phenotypes. Thus, in some implementations, the plurality of T cell phenotypes included in the QSP model 1202 are only and/or all of the stem-like memory T cell 210, the central memory T cell 212, the effector memory T cell 214, the effector T cell 216, and/or the endogenous T cell 219. In other implementations, the plurality of T cell phenotypes included in the QSP model 1202 are only and/or all of the stem-like memory T cell 210, the central memory T cell 212, the effector memory T cell 214, and the effector T cell 216.

In some implementations, the TCT product includes a living drug. In other words, the TCT product includes live and phenotypically diverse T cells. Thus, the actual composition and/or proportion of each of the plurality of T cell phenotypes that make up the TCT product may vary from patient to patient and/or between doses delivered to the same patient. The TCT product delivered to each patient may have a dose including a quantity of T cells. The dose of the TCT product may vary based on the patient. Additionally and/or alternatively, the dose of the TCT product may be delivered to the patient multiple times as part of a treatment regimen. Each dose delivered to the patient may be the same and/or vary. In other words, each dose may have a dose level including the same quantity of T cells or a different quantity of T cells. For example, the dose may include a dose of approximately 109 T cells, 10×109 T cells, 100×109 T cells, and/or the like. Thus, there may be a tenfold difference in a quantity of T cells included in each dose level corresponding to each dose.

The TCT product may be delivered to the patient after a lymphodepletion regimen is administered to the patient. This may help to prolong the persistence of plurality of T cell phenotypes of the TCT product delivered to the patient and increase the effectiveness of the delivered T cell therapy.

Referring to FIG. 2, the architecture 200 of the QSP model 1202 includes a plurality of physiological compartments. For example, the architecture 200 includes a peripheral tissue and lymph node compartment (i.e., healthy tissue compartment) 202, a blood compartment 204, a tumor draining lymph node compartment 208, and a tumor compartment 206. The tumor compartment 206 may include a tumor site of a tumor configured to be treated by the TCT product. The particular physiological compartments included in the architecture 200 were selected to improve the accuracy of predictions generated based on the QSP model 1202, to more accurately track T cell behavior within and between the plurality of physiological compartments, and to more accurately determine distributions of the plurality of T cell phenotypes. Thus, in some implementations, the plurality of physiological compartments included in the QSP model 1202 are only and/or all of the peripheral tissue and lymph node compartment (i.e., healthy tissue compartment) 202, the blood compartment 204, the tumor draining lymph node compartment 208, and the tumor compartment 206.

When the TCT product is delivered to the patient, the plurality of T cell phenotypes traffic to each of the plurality of physiological compartments. For example, in some embodiments, at least some or all of the plurality of physiological compartments includes the plurality of T cell phenotypes. For example, the peripheral tissue and lymph node compartment (i.e., healthy tissue compartment) 202, the blood compartment 204, the tumor draining lymph node compartment 208, and the tumor compartment 206 may include the stem-like memory T cell 210, the central memory T cell 212, the effector memory T cell 214, the effector T cell 216, and/or the endogenous T cell 219.

In some embodiments, each T cell phenotype of the plurality of T cell phenotypes is associated with a set of cellular kinetics parameters which describe the behavior of the T cell phenotypes after delivery of the TCT product to the patient. The set of cellular kinetics parameters includes one or more cellular kinetics parameters. For example, the set of cellular kinetics parameters includes at least one of a quantity, a proliferation (e.g., expansion) rate, an apoptosis rate, and a differentiation rate of the plurality of T cell phenotypes. The values for each cellular kinetics parameter of the one or more cellular kinetics parameters corresponding to each T cell phenotype of the plurality of T cell phenotypes within each physiological compartment may be different from one another.

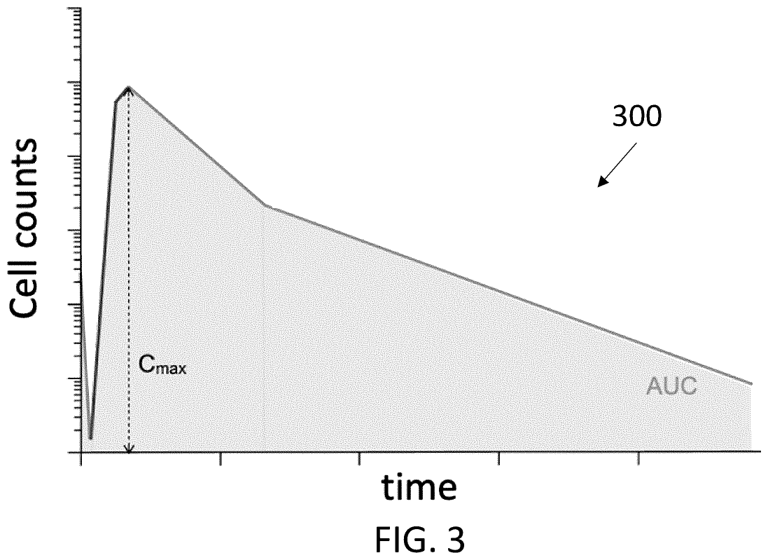

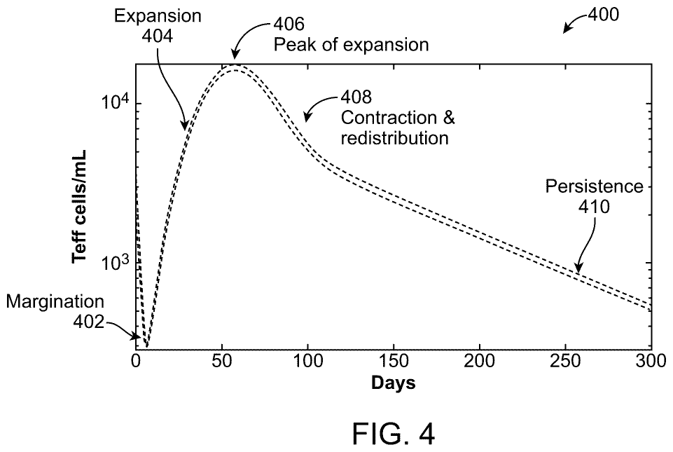

As an example, FIG. 3 illustrates a graph 300 showing T cell counts over time, consistent with implementations of the current subject matter. As shown in the graph 300, after delivery of the TCT product to the patient, a quantity of the T cells decreases before rapidly expanding. After reaching the maximum quantity of T cells, the quantity of T cells decreases slowly over time. As another example, FIG. 4 depicts an example graph 400 illustrating an effect of a set of cellular kinetics parameters for a T cell phenotype over time, consistent with implementations of the current subject matter. In particular, the graph 400 shows a concentration of an effector T cell over time after delivery of the TCT product the patient. For example, the graph 400 shows T cell behaviors, including trafficking (e.g., margination), proliferation (e.g., expansion), peak of proliferation (e.g., peak of expansion), apoptosis and redistribution, and persistence, over time, for the effector T cell in a physiological compartment after delivery of the TCT product in the patient. The set of cellular kinetics parameters for the T cell phenotype impact the behavior of the corresponding T cell phenotype over time, as exhibited by the graph 400. While the graph 400 illustrates the T cell behaviors corresponding to an effector T cell over time, any of the T cell phenotypes described herein may also experience the T cell behaviors, including trafficking (e.g., margination), proliferation (e.g., expansion), peak of proliferation (e.g., peak of expansion), apoptosis and redistribution, and persistence that may be represented by the set of cellular kinetics parameters (e.g., a change in the set of cellular kinetics parameters over time).

As an example, referring to graph 400, after delivery of the TCT product, the effector T cell undergoes margination 402 followed by rapid proliferation 404 as the concentration of effector T cells increases before reaching a peak of expansion 406. In some implementations, a rate of proliferation, the peak of expansion 406, and/or the like may indicate an efficacy of the T cell therapy. After reaching the peak of expansion 406, the effector T cell experiences apoptosis and redistribution 408 as the effector T cell traffics between physiological compartments and experiences apoptosis. Finally, the effector T cell experiences persistence 410. The persistence 410 (e.g., a length of persistence, and/or the like) may additionally and/or alternatively indicate an efficacy of the T cell therapy.

As noted, the set of cellular kinetics parameters may include a differentiation of the plurality of T cell phenotypes. In some implementations, as shown in FIG. 2, the architecture 200 includes differentiation of the effector memory T cell 214 into the effector T cell 216, at 240, after the effector memory T cell 214 has migrated into the tumor compartment 206. Differentiation of the effector memory T cell 214 into the effector T cell 216 within the tumor compartment can increase a quantity of the effector T cells for infiltrating the tumor 218 at 242. Further, the architecture 200 may include differentiation of the plurality of T cell phenotypes within the tumor draining lymph node compartment 208. Within the tumor draining lymph node compartment 208, the architecture 200 includes antigen-driven differentiation, at 244, of the stem-like memory T cell 210 into the central memory T cell 212, differentiation, at 246, of the central memory T cell 212 into the effector memory T cell 214, and differentiation, at 248, of the effector memory T cell 214 into the effector T cell 216. Activation of the plurality of T cell phenotypes within the tumor draining lymph node compartment 208 may be triggered by antigen-presenting cells on which antigens from the tumor are presented. The antigen-presenting cells activate the plurality of T cell phenotypes within the tumor draining lymph node compartment 208. This allows for the effector T cells 216 to recognize the tumor 218 and infiltrate the tumor 218.

Referring back to FIG. 2, the architecture 200 of the QSP model 1202 may include trafficking of the plurality of T cell phenotypes between each of the plurality physiological compartments. For example, the architecture 200 may include trafficking (e.g., via a trafficking rate or a margination rate) of the plurality of T cell phenotypes between the peripheral tissue and lymph node compartment (i.e., healthy tissue compartment) 202 and the blood compartment 204, between the blood compartment 204 and the tumor draining lymph node compartment 208, and between the blood compartment 204 and the tumor compartment 206. In other words, the architecture 200 may include trafficking (e.g., via a trafficking rate or a margination rate) of the plurality of T cell phenotypes from the peripheral tissue and lymph node compartment (i.e., healthy tissue compartment) 202 to the blood compartment 204, from the blood compartment 204 to the peripheral tissue and lymph node compartment (i.e., healthy tissue compartment) 202, from the blood compartment 204 to the tumor draining lymph node compartment 208, from the tumor draining lymph node compartment to the blood compartment 204, and/or from the blood compartment 204 to the tumor compartment 206.

In particular, as shown in FIG. 2, the architecture 200 includes trafficking of the stem-like memory T cell 210 between the peripheral tissue and lymph node compartment (i.e., healthy tissue compartment) 202 and the blood compartment 204, at 220, and trafficking of the stem-like memory T cell 210 between the blood compartment 204 and the tumor draining lymph node compartment 208, at 222. The architecture 200 may additionally and/or alternatively include trafficking of the central memory T cell 212 between the peripheral tissue and lymph node compartment (i.e., healthy tissue compartment) 202 and the blood compartment 204, at 224, and trafficking of the central memory T cell 214 between the blood compartment 204 and the tumor draining lymph node compartment 208, at 226. The architecture 200 may additionally and/or alternatively include trafficking of the effector memory T cell 214 between the peripheral tissue and lymph node compartment (i.e., healthy tissue compartment) 202 and the blood compartment 204, at 228, trafficking of the effector memory T cell 214 between the blood compartment 204 and the tumor draining lymph node compartment 208, at 230, and trafficking of the effector memory T cell 214 between the blood compartment 204 and the tumor compartment 206. The architecture 200 may additionally and/or alternatively include trafficking of the effector T cell 216 between the peripheral tissue and lymph node compartment (i.e., healthy tissue compartment) 202 and the blood compartment 204, at 232, trafficking of the effector T cell 216 from the blood compartment 204 to the tumor compartment 206, and from the tumor draining lymph node compartment 208 to the blood compartment 204. As shown in FIG. 2, the architecture 200 may additionally and/or include trafficking of the endogenous T cell 219 between the peripheral tissue and lymph node compartment (i.e., healthy tissue compartment) 202 and the blood compartment 204, and trafficking of the endogenous T cell 219 between the blood compartment 204 and the tumor draining lymph node compartment 208.

Again referring to FIG. 2, the architecture 200 includes proliferation (e.g., a proliferation rate, such as an antigen-driven proliferation rate and/or a homeostatic proliferation rate) for each of the plurality of T cell phenotypes within each of the physiological compartments (e.g., the peripheral tissue and lymph node compartment (i.e., healthy tissue compartment) 202, the blood compartment 204, the tumor compartment 206, and the tumor draining lymph node compartment 208).

For example, within the peripheral tissue and lymph node compartment (i.e., healthy tissue compartment) 202, the architecture 200 includes a proliferation rate (e.g., a homeostatic proliferation rate) 250 corresponding to the stem-like memory T cell 210, a proliferation rate (e.g., a homeostatic proliferation rate) 252 corresponding to the central memory T cell 212, a proliferation rate (e.g., a homeostatic proliferation rate) 254 corresponding to the effector memory T cell 214, and a proliferation rate (e.g., a homeostatic proliferation rate) 256 corresponding to the effector T cell 216. Within the blood compartment, the architecture 200 includes a proliferation rate (e.g., a homeostatic proliferation rate) 258 corresponding to the stem-like memory T cell 210, a proliferation rate (e.g., a homeostatic proliferation rate) 260 corresponding to the central memory T cell 212, a proliferation rate (e.g., a homeostatic proliferation rate) 262 corresponding to the effector memory T cell 214, and a proliferation rate (e.g., a homeostatic proliferation rate) 264 corresponding to the effector T cell 216. Within the tumor draining lymph node compartment 208, the architecture 200 includes a proliferation rate (e.g., an antigen-driven proliferation rate) 266 corresponding to the stem-like memory T cell 210, a proliferation rate (e.g., an antigen-driven proliferation rate) 268 corresponding to the central memory T cell 212, a proliferation rate (e.g., an antigen-driven proliferation rate) 270 corresponding to the effector memory T cell 214, and a proliferation rate (e.g., a homeostatic proliferation rate) 272 corresponding to the effector T cell 216. Within the tumor compartment 206, the architecture 200 includes a proliferation rate (e.g., a antigen-driven proliferation rate) 274 corresponding to the effector memory T cell 214, and a proliferation rate (e.g., a homeostatic proliferation rate) 276 corresponding to the stem-like memory T cell 216.

Again referring to FIG. 2, the architecture 200 includes apoptosis (e.g., an apoptosis rate) for each of the plurality of T cell phenotypes within each of the physiological compartments (e.g., the peripheral tissue and lymph node compartment (i.e., healthy tissue compartment) 202, the blood compartment 204, the tumor compartment 206, and the tumor draining lymph node compartment 208). For example, within the peripheral tissue and lymph node compartment (i.e., healthy tissue compartment) 202, the architecture 200 includes an apoptosis rate 278 corresponding to the stem-like memory T cell 210, an apoptosis rate 280 corresponding to the central memory T cell 212, an apoptosis rate 282 corresponding to the effector memory T cell 214, and an apoptosis rate 284 corresponding to the effector T cell 216. Within the blood compartment, the architecture 200 includes an apoptosis rate 286 corresponding to the stem-like memory T cell 210, an apoptosis rate 288 corresponding to the central memory T cell 212, an apoptosis rate 290 corresponding to the effector memory T cell 214, and an apoptosis rate 292 corresponding to the effector T cell 216. Within the tumor draining lymph node compartment 208, the architecture 200 includes an apoptosis rate 294 corresponding to the stem-like memory T cell 210, an apoptosis rate 296 corresponding to the central memory T cell 212, an apoptosis rate 298 corresponding to the effector memory T cell 214, and an apoptosis rate 299 corresponding to the effector T cell 216. Within the tumor compartment 206, the architecture 200 includes an apoptosis rate 297 corresponding to the effector memory T cell 214, and an apoptosis rate 295 corresponding to the stem-like memory T cell 216.

As noted above, the QSP model 1202 may be calibrated on clinical data. In some implementations, the cellular kinetics parameters, such as the quantity, the proliferation rate, the apoptosis rate, the differentiation rate, the trafficking rate, and/or the like, as described herein, may be determined based at least on the clinical data. The calibrated QSP model 1202 was able to recapitulate the kinetics of a plurality of T cell phenotypes targeting E7 in epithelial cancer patients dosed at three different dose levels (˜109, ˜1010, ˜1011 cells). The QSP model 1202 successfully captured the initial margination or trafficking, subsequent proliferation, apoptosis, and eventual persistence, among other cellular kinetics parameters for each of the T cell phenotypes observed across time for all three dose levels. Following calibration, the parameter space was sampled to create a virtual cohort that captured the variability of the clinical data across each dose level (described in more detail below). Additionally, the architecture 200 may be leveraged to determine the relationship between various cellular kinetics parameters, such as the persistence of T cells across time and the variability in cellular proliferation rates, differentiation rates, and trafficking rates of T cells. Finally, the architecture 200 may provide the impact of variability in dose level and phenotypic makeup of the initial TCT product on the cellular kinetics, distribution, and persistence of the T cell therapy across time.

In some implementations, the model generation engine 122 may determine a behavior of the plurality of T cell phenotypes within each of the plurality of physiological compartments (e.g., the peripheral tissue and lymph node compartment (i.e., healthy tissue compartment) 202, the blood compartment 204, the tumor compartment 206, and the tumor draining lymph node compartment 208, at various time points, at various doses (e.g., dose levels) of the TCT product, and/or at various compositions of the TCT product. In some implementations, the model generation engine 122 may determine a distribution of the corresponding T cell phenotypes of plurality of T cell phenotypes in each of the physiological compartments over time, such as based at least on the determined behavior of the plurality of T cell phenotypes within each of the plurality of physiological compartments at the various time points and between the plurality of physiological compartments at the various time points. In some implementations, the model generation engine 122 may determine a persistence of the corresponding T cell phenotypes of plurality of T cell phenotypes in each of the physiological compartments over time, such as based at least on the determined behavior of the plurality of T cell phenotypes within each of the plurality of physiological compartments at the various time points and between the plurality of physiological compartments at the various time points and/or based on the determined distribution.

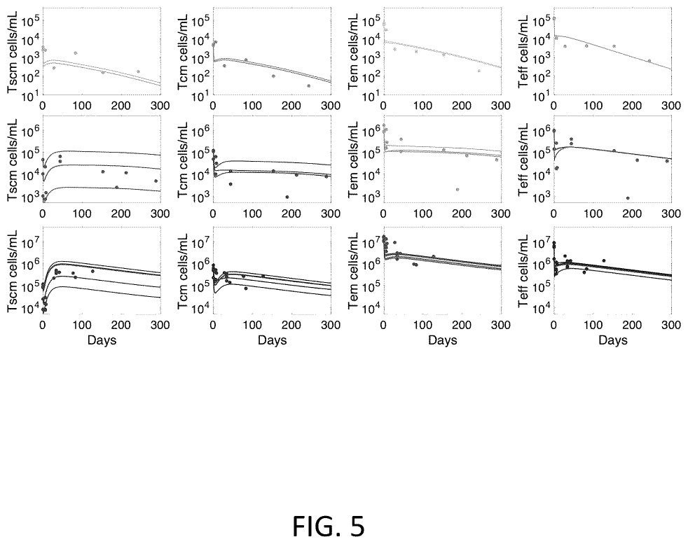

For example, the analysis engine 110 or model generation engine 122 may determine distributions or patient profiles (including sets of cellular kinetics parameters, patient responses, and/or the like) for each patient based on the determined behavior of the plurality of T cell phenotypes within and between the physiological compartments. FIG. 5 depicts an example comparison of cellular pharmacokinetics in blood, consistent with implementations of the current subject matter. In particular, FIG. 5 shows a comparison of behaviors of the plurality of T cell phenotypes over time (captured at various time points) across three doses and various patients. As noted herein, while a composition of the TCT product delivered to each patient was held constant, a constant composition of the TCT product may have some degree of variation (e.g., within a threshold range) at least because the TCT product may include a live drug. Further, a variation in the type of patient or the patient make-up may also impact variability in the results (as shown in FIG. 5). However, by varying the dose and incorporating a constant composition, the impact of the dose level on the cellular pharmacokinetics can be captured and determined by the analysis engine 110 or the model generation engine 122, such as based on the architecture 200. The architecture 200 may also account for such variations in the “constant” compositions of the TCT product and/or make-up of the patients.

For example, in FIG. 5, the first row of graphs corresponds to a low dose of the TCT product delivered to a first group of patients including two patients. The low dose included a composition of approximately 109 T cells. The second row of graphs corresponds to a middle dose of the TCT product delivered to a second group of patients including three patients. The middle dose included a composition of approximately 10×109 T cells. The third row of graphs corresponds to a high dose of the TCT product delivered to a third group of patients including five patients. The high dose included a composition of approximately 100×109 T cells. Thus, the various dose levels (low dose, middle dose, high dose, etc.) corresponds to a quantity of T cells and/or concentration of T cells in the TCT product delivered to the patient. Each line in the graphs corresponds to a different patient. As noted, the variability in the behavior of each of the T cells for each patient in response to delivery of the TCT product at each dose level is due at least in part to the variability in the constant composition of the TCT product and the particular make-up of the patient. In FIG. 5, the first column corresponds to a behavior of the stem-like memory T cell 210, the second column corresponds to a behavior of the central memory T cell 212, the third column corresponds to a behavior of the effector memory T cell 214, and the fourth column corresponds to a behavior of the effector T cell 216.

The distributions for each of the patients at each dose level may allow for determination of key drivers of cellular population values across time, which in turn may be used by the analysis engine 110 or the model generation engine 122 and/or as part of the architecture 200 to accurately determine doses and/or compositions of the TCT product to treat a particular patient as part of a T cell therapy, such as a T cell receptor (TCR)-engineered T cell therapy, an autologous T cell therapy, an allogeneic T cell therapy, an iPSC-derived T cell therapy, a CAR T cell therapy, or engineered T cell therapies w/TCRs targeting various tumor antigens. For example, the determined distributions and/or patient profiles described herein may allow for determination of an impact in variability in one or more of the set of cellular kinetics parameters and/or trafficking described herein on effector T cell counts. This may in turn allow for determination of an efficacy of a particular treatment. In some implementations, cellular kinetics parameters, such as proliferation and trafficking rates of the stem-like memory T cell 210 and/or trafficking, proliferation, and differentiation rates of effector memory T cells 214 may be key drivers for accurately determining and capturing patient profiles, such as those with high levels of cellular proliferation or decline. Accordingly, based at least on the architecture 200, the analysis engine 110 or the model generation engine 122 may predict the blood and tissue distribution and pharmacokinetics of T cells (e.g., the plurality of T cell phenotypes) following T cell therapy administration in patients with solid tumors. The analysis engine 110 or the model generation engine 122 may also leverage the architecture 200 to capture observed pharmacokinetics and efficacy of T cells (e.g., the plurality of T cell phenotypes) in preclinical animal species, such as mice to aide translational efforts of T cell therapies described herein.

In some embodiments, as described herein elsewhere in connection with FIGS. 2-5, the QSP model 1202 may be developed to simulate the dynamic profiles of various T cell phenotypes in the context of cell therapy treatments. In some embodiments, the QSP model 1202 may be meticulously designed to align with the relevant theoretical underpinnings of biological processes. As such, the QSP model 1202 may encapsulate the fundamental biomedical mechanisms governing T cell biology within physiologically significant compartments, encompassing both the native T cells within a patient and the TCR-engineered T cells introduced as part of the therapy. These compartments comprise key areas within the patient's body, including the central blood compartment, healthy tissues, tumor compartments, and tumor-draining lymph node compartments. The model 1202 may integrate essential biological mechanisms that are intrinsic to T cell behavior, such as antigen-induced proliferation and differentiation, cellular migration and trafficking, and the pivotal processes of homeostatic proliferation and apoptosis. The QSP model 1202 may be formulated using a system of ordinary differential equations (ODEs), which serve as the mathematical foundation for describing and forecasting the in vivo dynamics of distinct T cell phenotypes. In some embodiments, these phenotypes may comprise stem-like memory T cells (Tscm), central memory T cells (Tcm), effector memory T cells (Tem), and effector T cells (Teff) originating from TCR-engineered T cell therapy, in addition to the endogenous T cells (Tendo) naturally present within the patient's system throughout the treatment regimen.

In some embodiments, the digital twins generation engine 124 may generate digital twins by aid of the QSP model 1202. For example, by utilizing the QSP model 1202, the digital twins generation engine 124 may generate patient-specific digital twins that replicate the circulating T cell dynamics documented in a clinical trial involving TCR-engineered T cells directed against E7 in patients afflicted with metastatic HPV-associated epithelial cancers. In some embodiments, the analysis of critical factors influencing cellular kinetics and disparities among these digital twins may provide the following conclusions: 1. Stem cell-like memory T cells (Tscm) may serve as a pivotal determinant of both T cell expansion and persistence; 2. Differences associated with Tscm may play a substantial role in accounting for the observed variations in cellular kinetics among patients. Details of the analyses using digital twins are described in further details herein elsewhere. By conducting virtual clinical trials through computational modeling using digital twins, it is anticipated that increasing the presence of Tscm in the administered product will enhance the durability of engineered T cells, potentially enabling the utilization of lower dosage levels. Additionally or alternatively, the present disclosure validates the broader applicability of the QSP model 1202, digital twins, and the insights regarding the significance of Tscm enrichment by forecasting kinetics for two patients with pancreatic cancer subjected to KRAS G12D targeted T cell therapy.

In some embodiments, the QSP model 1202 characterizes the cellular kinetics of T cells within the bloodstream, various tissues, tumor-draining lymph nodes, and tumor environments, encompassing five distinct T cell phenotypes. In some embodiments, model calibration was conducted using clinical data from E7-targeting TCR-engineered T cells to generate a ‘reference virtual patient.’ Subsequently, in some embodiments, utilizing the same dataset, the digital twins generation engine 124 may facilitate the generation of a collection of digital twins, with each patient from the clinical trial matched to a subset of specific digital twins by, for example, the matching engine 126. For example, using the model 1202, the digital twins generation engine 124 may generate a few hundreds, a few thousands or more digitals twins. In some embodiments, each of the plurality of digital twins may represent a distribution of the plurality of T cell phenotypes in the plurality of physiological compartments over time associated with a corresponding patient, wherein the corresponding patient may be associated with a set of patient characteristics. In some embodiments, the matching engine 126 may receive a set of target patient characteristics, and may match the target patient characteristics with the patient characteristics associated with digital twins to select a subset of digital twins that resemble the target patient from the plurality of digital twins. In some embodiments, the matching engine 126 may compute matching scores for each digital twin within the collection concerning the target patient. Subsequently, the matching engine 126 may select the top N digital twins with the highest scores. This may facilitate a selection of a subset of digital twins from the collection of digital twins that resemble the target patient among the available digital twins.

In some embodiments, a set of patient characteristics, comprising patient biometrics, patient medical history, and baseline biomarker data, may be utilized as the basis for computing a matching score. This matching score is utilized to assess and determine the similarity between the target patient and a collection of digital twins. The matching score calculation may comprise different methodologies, for example, biometric-driven scoring, medical history-driven scoring, biomarker data-driven scoring, integrated scoring considering all characteristics, weighted scoring based on characteristic relevance, dynamic adjustment of scoring criteria, and threshold-based scoring. These examples provide versatile approaches to facilitate the selection of digital twins that closely align with the target patient's characteristics across a spectrum of clinical scenarios, and may enable making predictive simulations for a patient's response.

In some embodiments, for each target patient, the matching engine 126 may select a few digital twins virtual patients, for example, 2, 3, 4, 5, 6, 7, 8, 9, 10, 11, 12, 13, 14, 15, 16, 17, 18, 19, 20, 25, 30, 35, 40, 45, 50 etc. In some embodiments, each of these selected digital twins may be crafted to represent a distinct combination of underlying biological parameter values, aiming to closely replicate the observed patient kinetics when subjected to the corresponding clinically administered dose amount and composition. Given the inherent uncertainty in biological parameters, the utilization of multiple digital twins per patient may provide a number of benefits, for example, it may enable explicit consideration of alternative parameterizations of the underlying biology, which may remain consistent with the observed data. Additionally or alternatively, this approach may accommodate scenarios where divergent predictions may arise under untested protocols, different dosing regimens, or variations in the cell composition of the administered product.

These digital twins have proven useful for making predictions concerning alternative dosing strategies, for example, by the treatment plan generation engine 128. In some embodiments, utilizing the selected subset of digital twins for the target patient, predictions are made regarding the target patient's responses to a multitude of hypothetical deliveries of the TCT product. These hypothetical deliveries encompass a range of distinct dose quantities and compositions of T cell phenotypes. Through this approach, comprehensive insights into potential responses and outcomes can be generated, aiding in informed decision-making for treatment strategies. Furthermore, in some embodiments, parameter analyses have been conducted to gain insights into the biological mechanisms that may diverge between individual patients or drive sustained cellular persistence over time. The digital twins, in some embodiments, have been subjected to simulation involving an alternative dosing strategy, thus enabling predictions regarding the influence of dose composition and dose quantity on cellular kinetics. In some embodiments, the predictions generated by the digital twins have been validated through a distinct TCR-engineered T cell therapy scenario, specifically targeting KRAS G12D in patients diagnosed with pancreatic cancer.

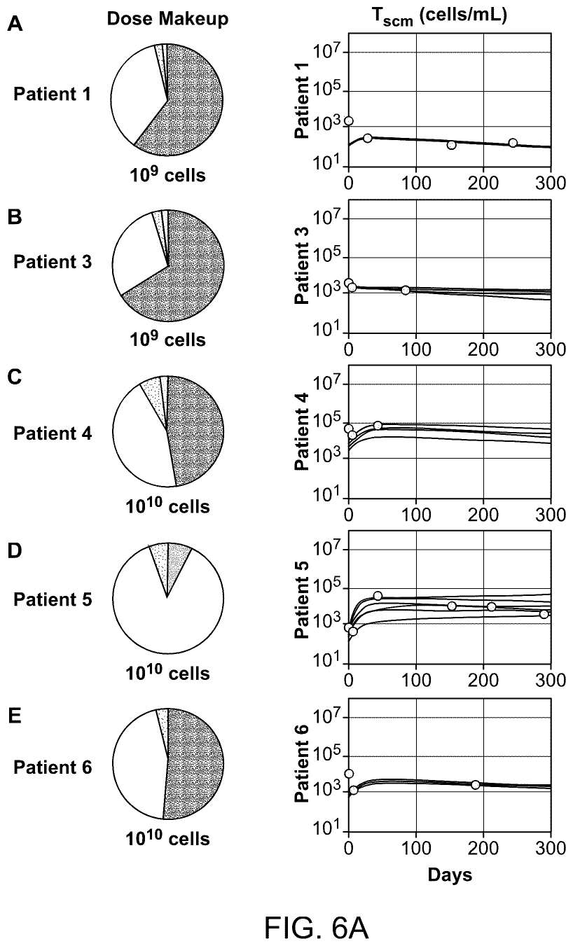

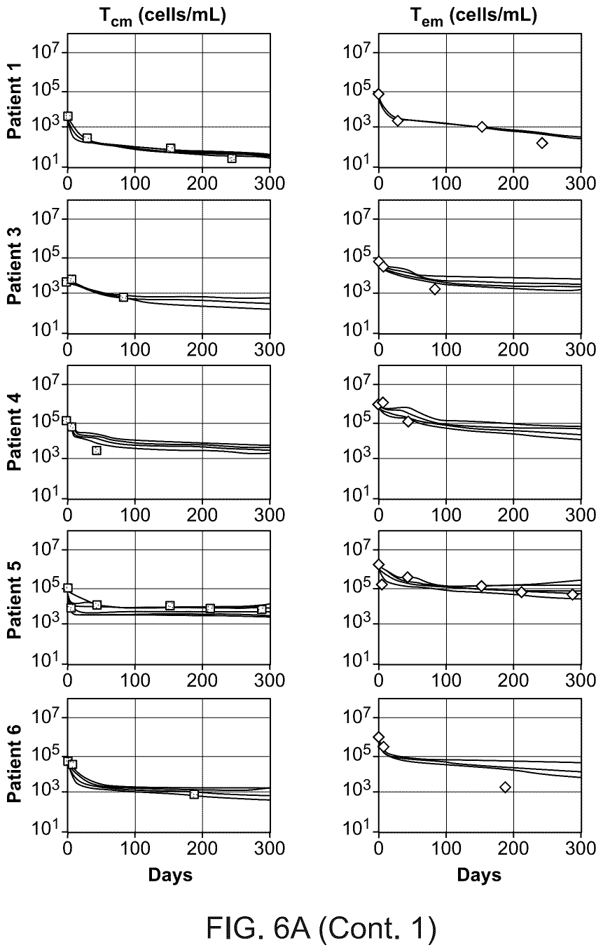

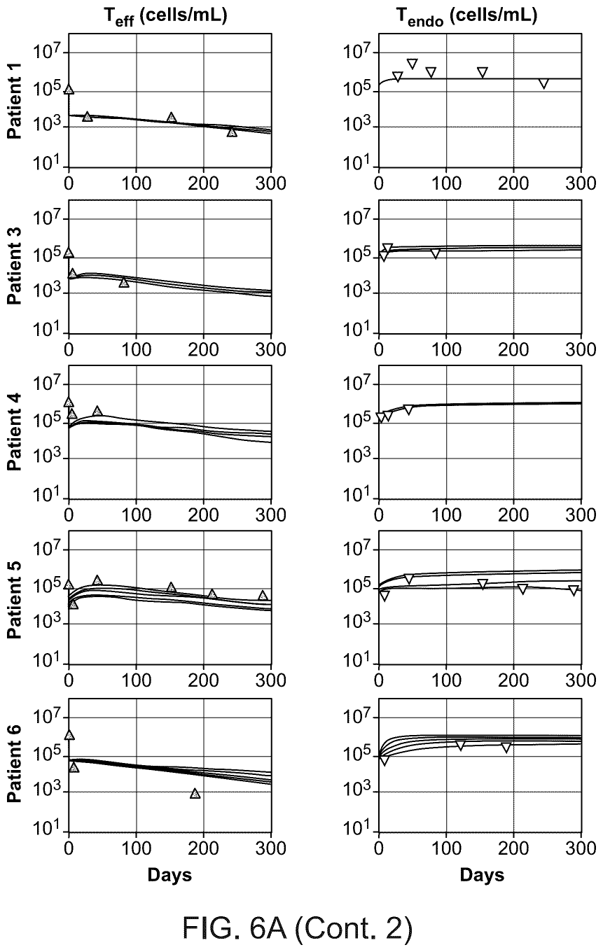

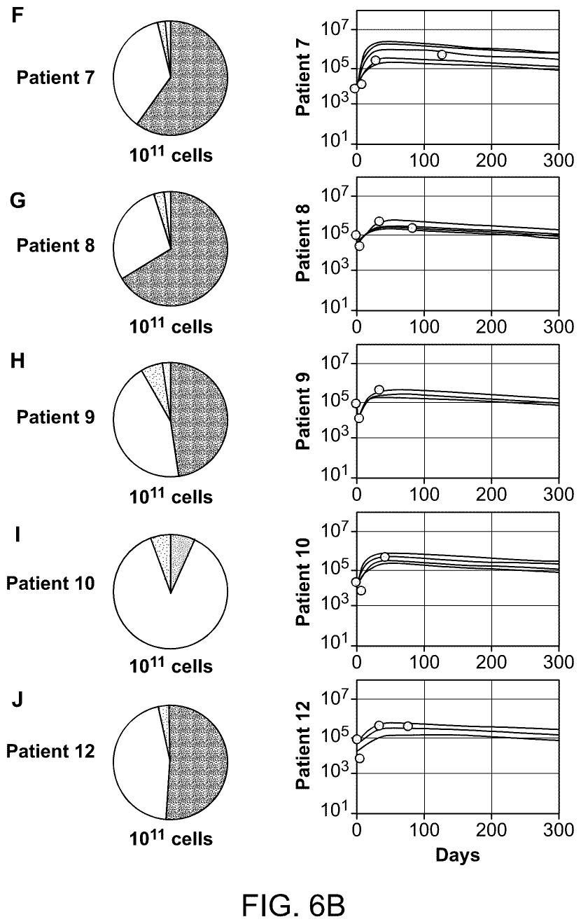

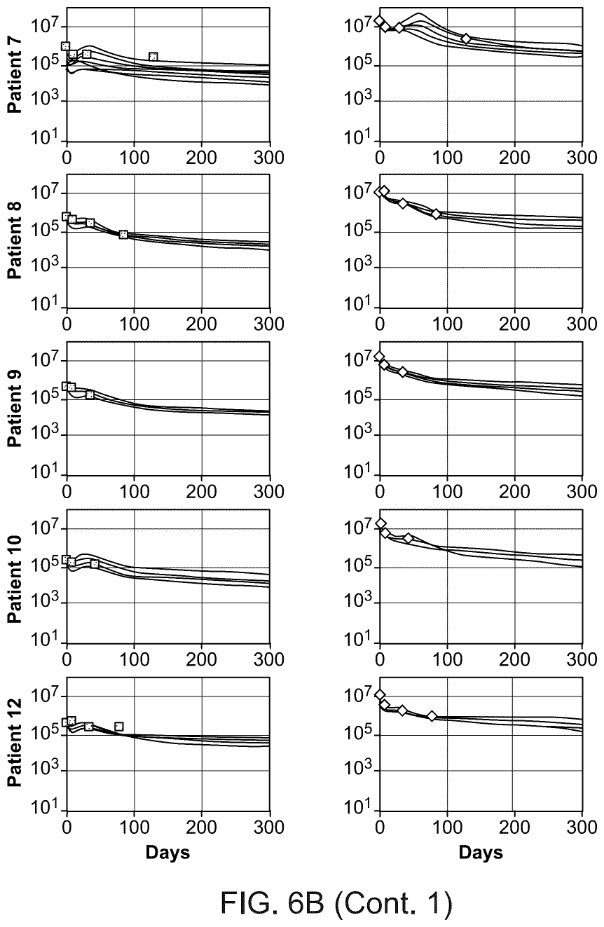

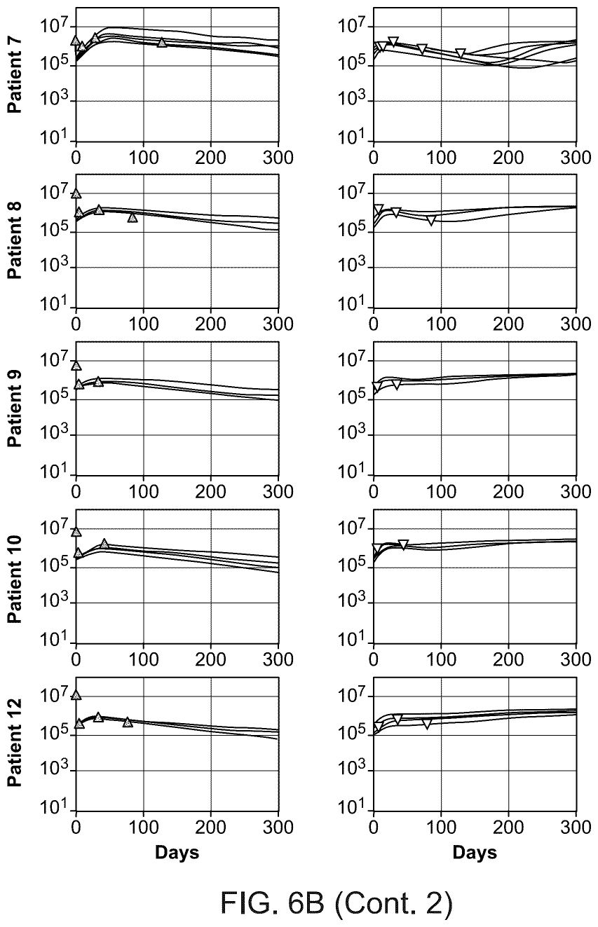

FIGS. 6A and 6B depict example comparison of cellular pharmacokinetics in blood across various dose groups and patients, consistent with implementations of the current subject matter. In particular, FIGS. 6A and 6B show individualized virtual patients characterizing variability across dose level groups, compositions of the TCT treatment, and patients. In FIGS. 6A and 6B, each row corresponds to a different patient and the graphs in each column correspond to a particular T cell phenotype. For example, the graphs in the first column correspond to the stem-like memory T cell 210, the graphs in the second column correspond to the central memory T cell 212, the graphs in the third column correspond to the effector memory T cell 214, the graphs in the fourth column correspond to the effector T cell 216, and the graphs in the fifth column correspond to the endogenous T cell. Each of the lines in each graph corresponds to a different dose (e.g., quantity and/or concentration of T cells) of the TCT product delivered to each patient, and a composition (e.g., the specific ratio or proportion of various T cell phenotypes, such as stem-like memory T cells, central memory T cells, effector memory T cells, effector T cells, and endogenous T cells) of the TCT product delivered to each patient.

Based on the architecture 200 of the QSP model 1202, the analysis engine 110 or the model generation engine 122 may determine the various distributions (e.g., the graphs representing patient profiles corresponding to each of the plurality of T cell phenotypes), as shown in FIGS. 6A and 6B. Based on the determined distributions (e.g., patient profiles), the analysis engine 110 or the model generation engine 122 may predict the dynamics of T cell subsets or T cell phenotype compositions for the TCT product and the impact of each composition or subset on anti-tumor efficacy over an extended period of time. Thus, the architecture 200 may allow for virtual clinical trials and/or experiments to be conducted, with aid of the digital twins. The architecture 200 may additionally and/or alternatively allow for the analysis engine 110 or the model generation engine 122 to determine, based on the architecture 200, the impact of variations in lymphodepletion efficiency and/or protocols on the anti-tumor efficacy, T cell phenotype compositions of the TCT product, and/or doses and regimens of T cell therapy administration.

Additionally and/or alternatively, based at least on the architecture 200 and/or the determined patient profiles for at least one, two, or more patients, the analysis engine 110 or the treatment plan generation engine 128 may predict individual patient responses (or ranges thereof) to a given TCT product by simulations based on cellular kinetics parameters determined by individual patient baseline biomarker data (data collected via genomic, transcriptomic, and/or flow cytometry methods). For example, the analysis engine 110 may determine the response in a target patient to the T cell therapy including the given TCT product by at least simulating, based at least on the architecture 200 and/or the determined patient profiles (e.g., distributions), a plurality of responses to a plurality of doses and/or a composition (or plurality of compositions) of the TCT product in a plurality of simulated patients (as shown in FIGS. 6A and 6B). The plurality of responses may be simulated by at least varying a dose, a composition, and/or the like of the TCT product.

The response in the target patient may be one of the plurality of simulated responses. For example, at least one of the simulated patient profiles (e.g., distributions, responses, etc.) corresponding to a patient having a similar characteristic, make-up, diagnoses, tumor, cancer, and/or the like as the target patient may be selected (e.g., by the analysis engine 110 or the matching engine 126). Based on the selected simulated patient profile, an effective T cell therapy including a dose and/or a composition of a TCT product may be determined and/or administered to the target patient to treat the tumor and/or cancer. This allows for determination of an ideal dose and/or composition of the TCT product for treating a tumor and/or cancer in the target patient. In some implementations, the simulated patient profiles can be used to evaluate both safety, such as with respect to cytokine release syndrome (CRS) and efficacy of a particular T cell therapy including a particular dose and/or composition of a TCT product and regimen.

Further, as shown in FIGS. 6A and 6B, the digital twins generation engine 124 may capture the distinct cellular kinetics exhibited by each patient in response to varying dose quantities and compositions of TCR-engineered T cells (FIGS. 6A and 6B, with patient IDs 2 and 11 excluded from the dataset due to data unavailability). The digital twins, akin to the reference patient, replicate the disparities observed between different patient cohorts. Notably, they may reproduce the limited cell expansion in patients belonging to the low-dose group (e.g., IDs 1 and 3) and the more substantial expansion of Tscm and Teff cell populations in patients within the high-dose cohort (e.g., IDs 7, 8, 9, 10, 12). Furthermore, the digital twins may capture the intragroup variability. For instance, within the middle dose group, the simulations emulate the variations in cell numbers and kinetic profiles between patient 5 and patients 4 and 6. The initial composition differences may be identified as a source of variability for the predicted patient responses.

As shown in FIGS. 6A and 6B, the digital twins form a virtual population that represents the 10 patients. This virtual population may be employed to predict cellular kinetics for these 10 patients under diverse TCR-engineered T cell therapy regimens and compositions, enabling a detailed dissection of the biological mechanisms illustrating the observed and predicted kinetics and the interpatient variability. Each column in the provided data displays experimental measurements in blood, encompassing Tscm, Tcm, Tem, or Teff TCR-engineered T cells, as well as endogenous T cells (Tendo). Each row presents cell measurements in units of cells per milliliter (cells/mL) for an individual patient. As shown in FIGS. 6A and 6B, Patients 1 and 3 received 10\hat{ }9 cells, patients 4, 5, and 6 received 10\hat{ }10 cells, and all other patients received 10\hat{ }11 cells. Data points correspond to experimental measurements over time, while the curves represent the 10 digital twins that exhibit the closest match to the experimental data for each respective patient. The digital twins shown in FIGS. 6A and 6B capture the variations in cellular kinetics among patients undergoing treatment with TCR-engineered T cells. Notably, they reproduce the multiphasic cellular kinetics observed in blood following the administration of HPV-16 E7-targeting TCR-engineered T cells.

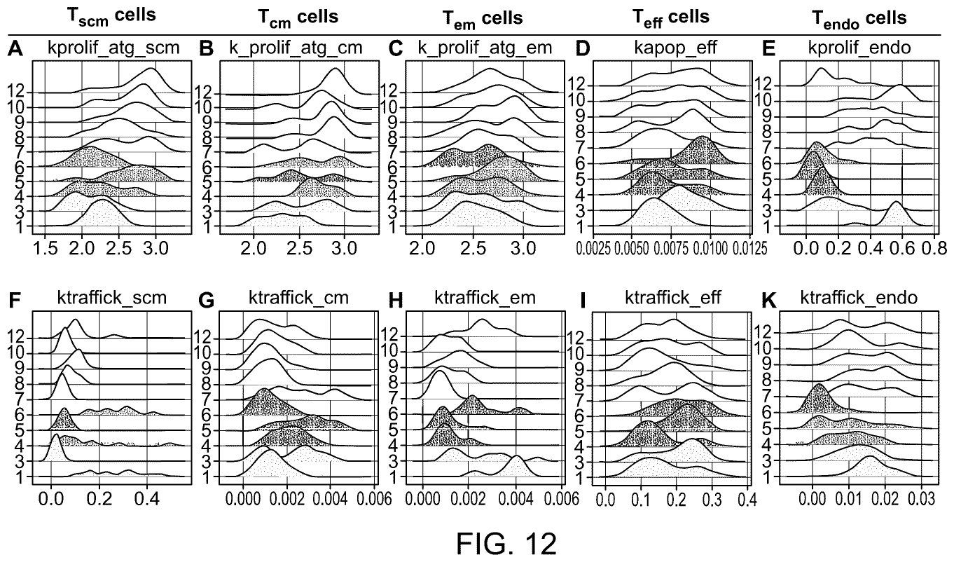

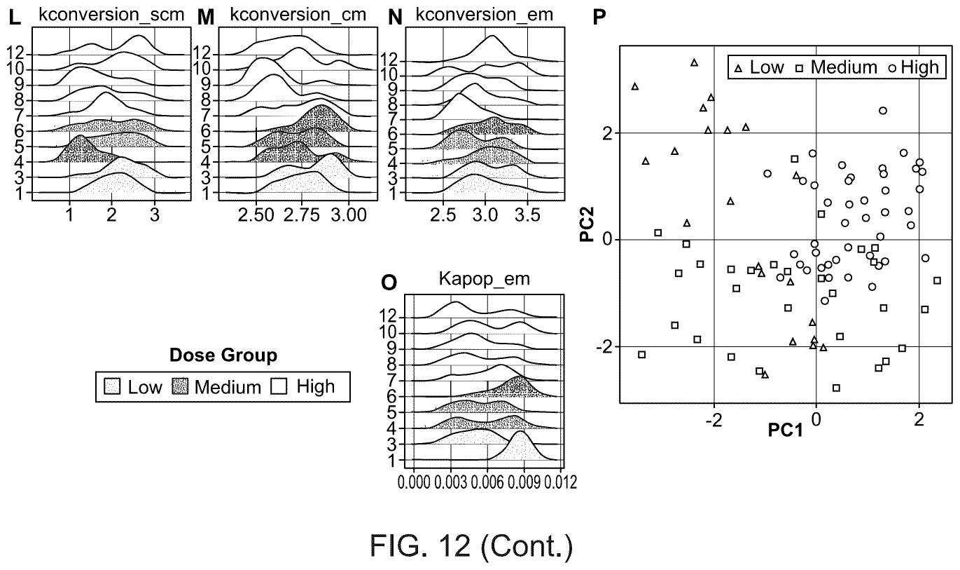

FIG. 12 depicts example parameter space ridgeline plots and principal component analysis of digital twins reveal sources of variability between patients and across dose groups (A-O). As shown in FIG. 12, ridgeline plots may visualize the distribution for 14 parameters (1 subplot per parameter) across the digital twins of each clinical patient (i.e., a target patient). As shown in FIG. 12, for each subplot (i.e., shown by FIG. 12A-FIG. 12O), individual patient IDs are listed along the y axis, and the range of potential parameter values are listed along the x axis. FIG. 12P illustrates variability across principal components of parameter space (PC1 vs PC2). In some embodiments, each dot in FIG. 12P may represent an individual digital twin, with a total of 100 digital twins represented.

The digital twins, characterized by distinct inferred parameterizations of the underlying biological processes governing cellular kinetics, aid in this analysis. Ridgeline plots as shown in FIG. 12A-FIG. 12O and principal component analysis (PCA) (FIG. 12P) serve for visualizing the parameter space for digital twins associated with each patient and facilitating cross-patient comparisons.

As shown in FIG. 12, several parameters exhibit consistent distributions across patients in the ridgeline plots (FIG. 12A-FIG. 12O). For instance, parameters related to antigen-driven Tem proliferation (FIG. 12C) and Tem conversion (FIG. 12N) display similar distributions, suggesting that these processes do not significantly contribute to interpatient differences in cellular kinetics. However, the rate constant governing antigen-driven proliferation of Tscm cells (i.e., kprolif_atg_scm subplot—FIG. 12A) demonstrates notable variability both within and across dose groups. Within the mid-dose group, digital twins for patient 5 exhibit higher Tscm proliferation rate constants, akin to values observed in the high-dose cohort. Conversely, digital twins for patients 4 and 6 feature lower Tscm proliferation rate constants, akin to values observed in the low-dose cohort. This observation aligns with the finding that patient 5 displays higher cell counts compared to patients 4 and 6, suggesting that the propensity for antigen driven Tscm proliferation plays a crucial role in interpatient variability in T cell numbers.

Alternatively or additionally, certain parameters and processes exhibit substantial interpatient variability, comprising the trafficking rate constants of Tscm, Tcm, and Tem (FIGS. 12F, 12G, 12H), as well as the proliferation rate constants of Tcm (FIG. 12B) and endogenous cells (FIG. 12E). These findings may underscore the significance of these biological processes as key determinants of cell kinetics and heterogeneity. Alternatively or additionally, the analysis reveals clear inter-cohort variability among specific parameters. This is further exemplified in a PCA plot (FIG. 12P), where minimal overlap is observed along the first principal component (PC1) between the low and high dose groups. In the PCA, parameters governing T cell proliferation and trafficking emerge as influential contributors to variability across dose cohorts, as substantiated by the contribution analysis. These trends suggest a complex interplay between dose levels and early memory T cell proliferation, indicating effects that extend beyond those explicitly incorporated into the model.

In some embodiments, as shown in FIG. 12, the identification of sources of variability across patients may be achieved by visualizing a parameter space for a subset of the plurality of digital twins. This analysis allows for a comprehensive assessment of potential underlying factors contributing to differential kinetic profiles among patients. Furthermore, the model 1202 may be adapted and/or modified to account for these identified sources of variability across patients.

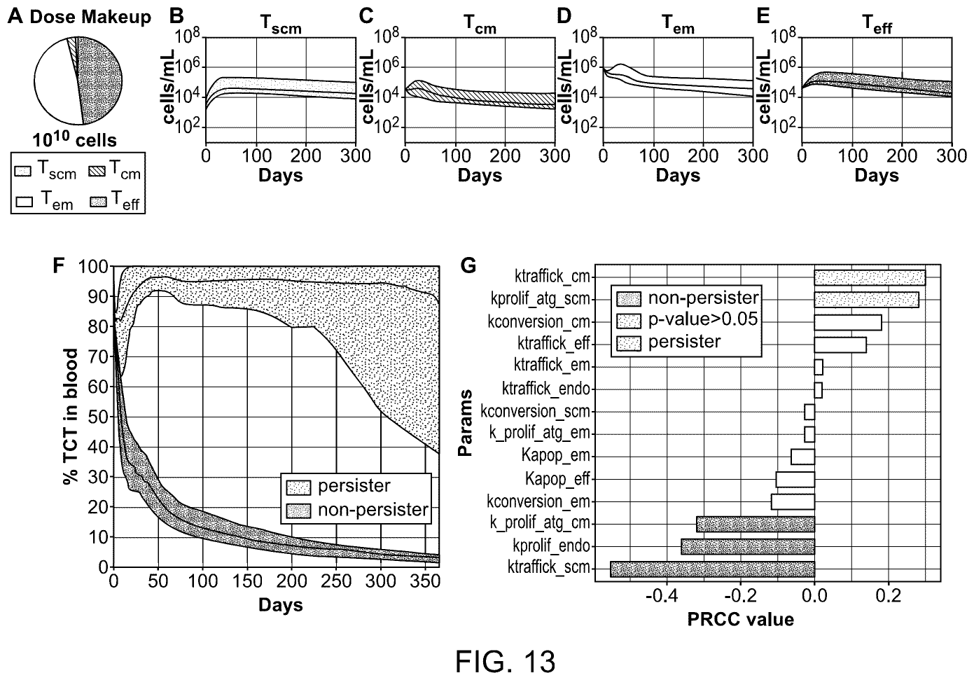

FIG. 13 depicts example biological variability (patient-specific) impacts cellular kinetics of TCR-engineered T cell therapies leading to persister or non-persister outcomes. As shown in FIG. 13, the collection of digital twins generated by the digital twins generation engine 124 may be simulated with a dose of 1010 TCR-engineered T cells and a dose composition of 1% Tscm, 3% Tcm, 48% Tem, and 48% Teff cells (the composition is illustrated by FIG. 13A). FIGS. 13B-13E illustrate the cellular kinetics of each T cell phenotype across time. In some embodiments, the bands depicted in the graph may represent the 25th and 75th percentiles of each subgroup, while the solid lines represent the median values for each T cell phenotype. Additionally or alternatively, FIG. 13G presents a bar plot illustrating the Partial Rank Correlation Coefficient (PRCC) scores. Each parameter is represented along the y-axis, and its relationship with the outcome (persister vs. non-persister) is quantified. Empty bars may signify parameters that were statistically insignificant in this context.

As shown in FIG. 13, the stratification of digital twins within a virtual clinical trial offers insights into the key determinants influencing T cell persistence. For example, as shown in FIG. 13, the preceding analysis may provide a visualization of the biological variability inherent across the digital twin population. In some embodiments, digital twins may enable the assessment of how interpatient biological variability impacts T cell persistence over time. As shown in the example depicted by FIG. 13, a virtual clinical trial was simulated in which all digital twins received a mid-dose comprising 10\hat{ }10 TCR-engineered T cells, with a composition consisting of 1% Tscm, 3% Tcm, 48% Tem, and 48% Teff cells (FIG. 13A), mirroring a representative dose composition from the E7 clinical trial. The simulated kinetics (median and interquartile range) of various T cell phenotypes across the virtual population are depicted in FIGS. 13B-13E. At this fixed dose amount and composition, both Tscm and Teff cells (FIG. 13B, FIG. 13E) exhibit initial distribution and expansion, followed by a subsequent decline across all digital twins. In contrast, Tcm and Tem cells (FIG. 13C, FIG. 13D) are predicted to undergo less expansion. Notably, the predicted responses span a range of nearly an order of magnitude for each T cell phenotype, underscoring the anticipated interpatient variability under a consistent dose composition.

Given the correlation between cell expansion in the initial months and treatment efficacy in hematological malignancies, a stratification of digital twins based on the frequency of TCR-engineered T cells in the blood at day 50 was performed. Those digital twins exhibiting a predominant presence of TCR-engineered T cells in the blood, indicative of persistence over time, were categorized as ‘persisters’ under this treatment regimen, while the rest, displaying a decline over time, were categorized as ‘non-persisters’ (FIG. 13F).

As shown in FIG. 13, to delve into the underlying biological processes driving persister vs. non-persister outcomes, a global sensitivity analysis may be conducted. In some embodiments, rate constants governing Tscm trafficking and proliferation, Tendo proliferation, and Tcm proliferation and trafficking were identified as key determinants of persister or non-persister outcomes (Partial Rank Correlation Coefficient values are illustrated in FIG. 13G). Notably, these parameters were also identified in the previous analysis of inter-individual variability. As such, non-linear relationships between parameters may exist. For instance, as overall Tscm expansion relies on both trafficking to and proliferation within the tumor-draining lymph node, digital twins with lower Tscm trafficking rate constants may require higher Tscm proliferation rate constants to exhibit persistence, and vice versa. These differences in parameters between persisters and non-persisters become more apparent in a bivariate plot. In some embodiments, these parameters emerge as key drivers of interpatient variability in cell persistence, which may indicate a necessity to optimize treatment regimens, dose, and/or composition to modulate these parameters effectively and achieve desired levels of T cell persistence.

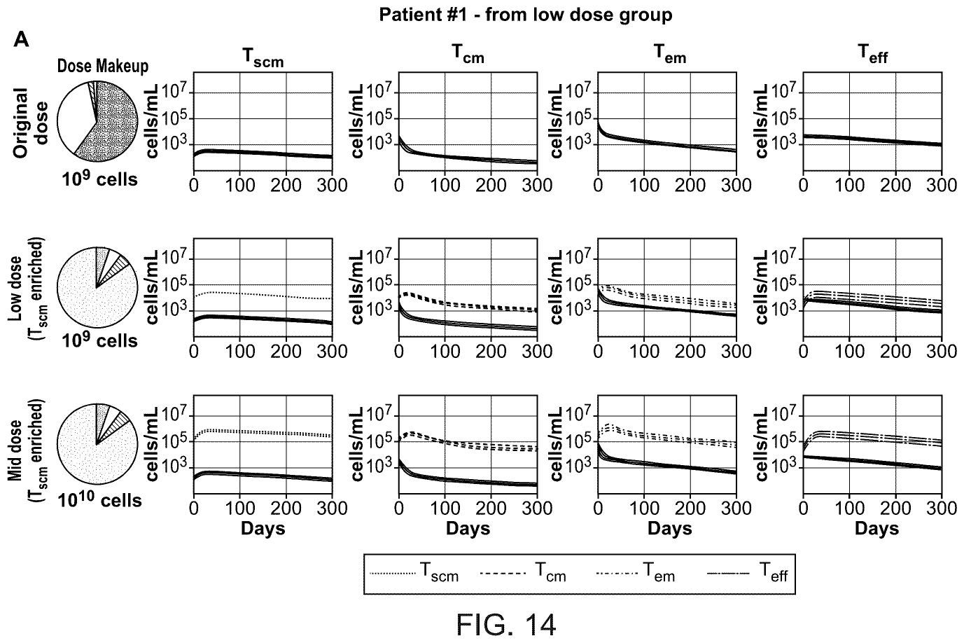

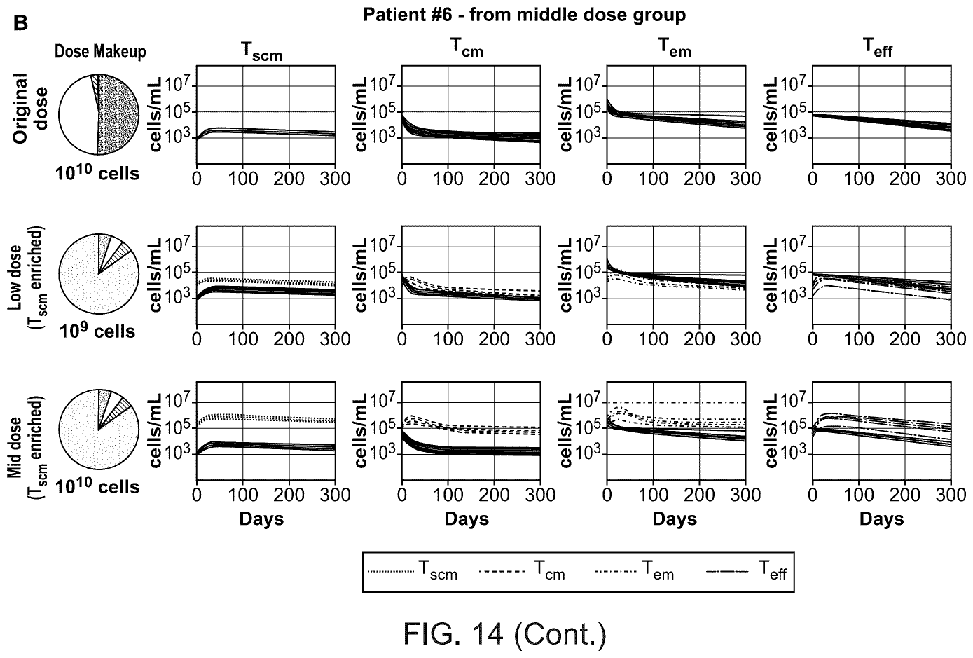

FIG. 14 depicts example graphs showing dose composition impacts cellular kinetics of TCR-engineered T cells. This figure presents two distinct sets of digital twins: one matching patient 1 (i.e., in connection with FIG. 14A) and the other matching patient 6 (i.e., in connection with FIG. 14B). In some embodiments, these digital twins may be re-simulated with alternative dose compositions and varying dose amounts. Each row within the figure may correspond to a different dose composition or dose amount that undergoes simulation. Each column may illustrate the representation of the dose composition or the cellular kinetics of the TCR-engineered T cell subsets. To facilitate comparison, the cellular kinetics of the digital twins treated with the original dose composition are replotted in the corresponding curves, serving as a baseline against which the cellular kinetics resulting from alternative dosing simulations.

In some embodiments, other than the dose quantity and inherent biological patient variability, dose composition may exert an influence on the expansion and persistence of T cells. To exemplify the implications of alternative dose compositions, simulations may be conducted with different compositions for representative patients drawn from both the low-dose (patient 1) and mid-dose (patient 6) cohorts (as shown by FIGS. 6A and 6B). Specifically, a composition enriched with Tscm cells (comprising 85% Tscm, 5% Tcm, 5% Tem, and 5% Teff) was used, based on the hypothesis that Tscm enrichment might facilitate more substantial expansion and prolonged persistence. The cellular kinetics following treatment with the original dose composition are compared with those predicted after treatment with the Tscm-enriched dose at two distinct dose levels, as shown in FIG. 14.

The predicted cellular kinetics may support the notion that Tscm-based treatments result in enhanced overall expansion and persistence of TCR-engineered T cells. For patient 6, as shown in FIG. 14, the simulated infusion of a Tscm-enriched product at a 10-fold lower dose (10\hat{ }9 cells) is projected to yield a comparable overall cell expansion and persistence compared to the infusion of 10\hat{ }10 cells of the original dosing material, primarily composed of Tem and Teff cells. In the case of patient 1, the simulated infusion of the Tscm-enriched material at the original dose level of 10\hat{ }9 cells predicts an approximately 100-fold increase in T cell counts in the bloodstream compared to the original dosing material, which primarily consists of Tem and Teff cells. This simulation, indicating that a higher Tscm composition in the infused product corresponds to greater T cell abundance in these patients, aligns with the earlier findings regarding the critical role of Tscm parameters in shaping cellular kinetics and persistence. Notably, these insights go beyond the scope of what can be gleaned from a mere correlation analysis of the E7 clinical trial. Therefore, in some embodiments, the treatment plan generate engine 128 may utilize the QSP model 1202 and digital twins to perform the optimization of dose composition and quantity for a clinical patient (i.e., a target patient), ultimately aiming to enhance T cell persistence in clinical applications.

In some embodiments, following the exploration of optimized dose composition and quantity using the QSP model and digital twins, these simulations and finding may be leveraged for a real-world clinical scenario. For example, the system may predict the responses of the target patient to a plurality of hypothetical deliveries of the TCT product, each delivery may be characterized by distinct combinations of T cell dose and composition. To predict this target patient responses, a subset of digital twins resembling the target patient's characteristics may be selected. These selected digital twins offer a predictive framework to estimate how the patient's T cell kinetics would respond to various treatment scenarios, thereby enabling a comprehensive evaluation of potential outcomes.

In some embodiments, the treatment plan generation engine 128 may utilize the predicted target patient responses provided by the subset of digital twins. As such, the treatment plan generation engine 128 may formulate an individualized treatment plan for the target patient, taking into account the predicted cellular kinetics and persistence levels in the various physiological compartments over time. This treatment plan is designed, for example, by the treatment plan generation engine 128, to optimize that the T cell persistence within the target patient, which may consistently exceed a predetermined threshold across multiple physiological compartments. In some embodiments, the treatment plan dictates the specific dosage and composition of the TCT product for the patient's therapy, aligning it with the anticipated goal of sustaining T cell levels above the defined threshold. Leveraging insights derived from the QSP model 1202 and digital twins, this application establishes a framework for data-driven, personalized decision-making within the domain of TCR-engineered T cell therapy. It introduces the capacity to fine-tune treatment approaches on a per-patient basis, with a focus on optimizing both the composition and quantity of the therapeutic T cell doses. The aim is to attain and sustain the targeted T cell persistence levels, ultimately bolstering the precision and efficacy of clinical interventions in this specialized field.

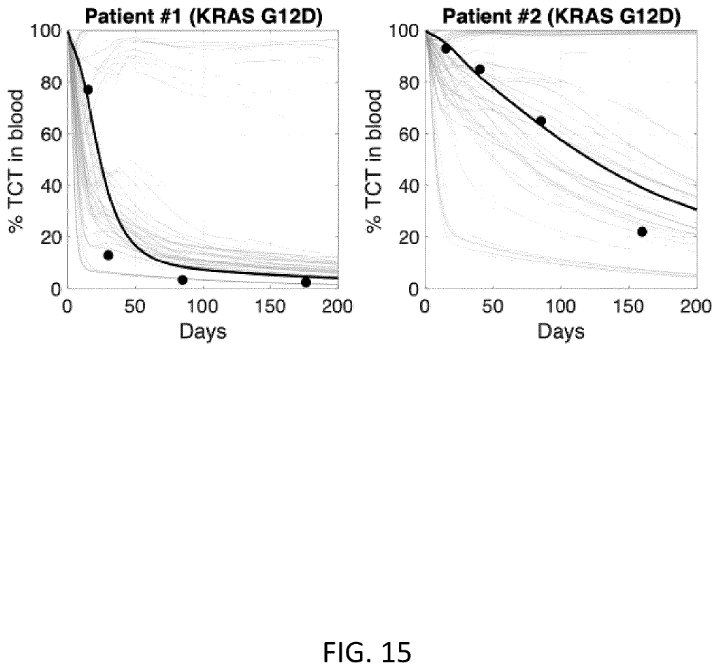

FIG. 15 depicts example predictive simulations of digital twins demonstrate alignment with observed profiles in patients with available clinical data. In some embodiments, clinical data regarding patients with pancreatic cancer treated with TCR-engineered T cells targeting KRAS G12D may be utilized. As shown in FIG. 15, the percentage of TCR-engineered T cells relative to total T cells in blood is depicted for FIG. 15A patient #1 and FIG. 15B patient #2 across time. As shown in FIG. 15, 100 digital twins (represented by lighter color curves) may be subjected to re-simulation with the dose amount and composition specified in the clinical trial for KRAS G12D. The darker color curves in FIG. 15 may represent the single digital twin that most accurately reproduces the observed data for each patient, as determined by the Root Mean Square Error (RMSE).

As shown in FIG. 15, the predictive capabilities of digital twins in forecasting T cell profiles for patients with KRAS G12D-targeting TCR-engineered T cell therapy is illustrated. In some embodiments, to assess the applicability of the QSP model structure and digital twins to other TCR-engineered T cell therapies and medical indications, simulations were conducted using the 100-member digital twin virtual population, employing the same dosage and composition parameters employed in a clinical study involving TCR-engineered T cells targeting KRAS G12D in metastatic pancreatic cancer patients. The selection of the KRAS G12D clinical dataset for this evaluation may be based on its public availability, offering insights into dose composition, dosage volume, and the dynamics of this therapy across different patients. In the context of the KRAS G12D clinical trial, both patients were administered an intermediate dose of approximately 1010 cells, primarily composed of Tem cells, although patient #2 received a larger proportion of early memory cells. As such, the variation in simulation curves may reflect the inherent inter-patient and intra-patient variability encapsulated within the digital twins. The trajectories of clinical patients align with the range of predictions made by the digital twins, with the best-matched digital twin in each instance converging with the densest cluster of simulated profiles. FIG. 15 additionally validates the model 1202 and the digital twins because it demonstrates their capacity to accurately predict the cellular kinetics of TCR-engineered T cells in patients with pancreatic cancer undergoing therapy targeting KRAS G12D. This alignment between predicted and observed profiles underscores the robustness and generalizability of the model and digital twins, extending their utility beyond the specific therapy and patient cohort studied, and suggests their potential applicability for forecasting T cell behavior in diverse TCR-engineered therapies and medical contexts.

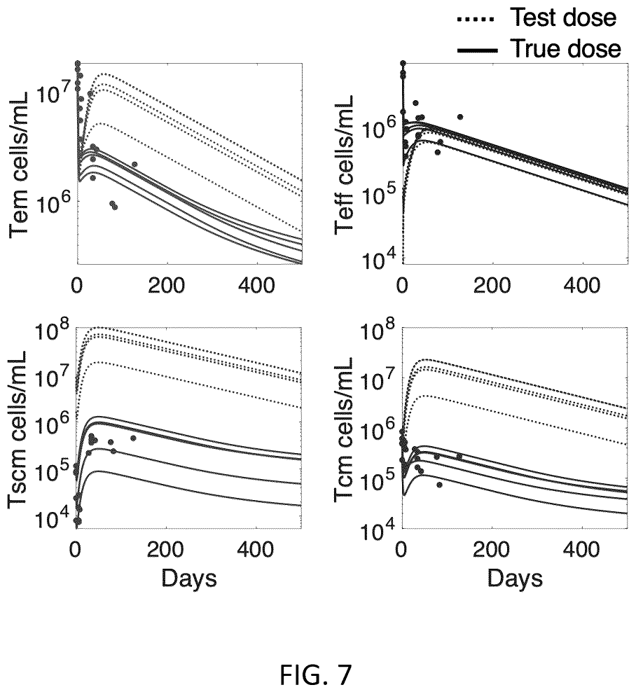

FIG. 7 depicts example graphs showing changes in cellular pharmacokinetics based on phenotypic makeup of a T cell product and dose level, consistent with implementations of the current subject matter. As noted, the architecture 200 allows for determination of the impact of variability in dose level and phenotypic makeup of the TCT product on the cellular pharmacokinetics of the plurality of T cell phenotypes, and the distribution and persistence of the T cell therapy across time. FIG. 7 includes graphs depicting a re-simulation of the high dose group of patients, for instance, the examples depicted by the third row of FIG. 5. The graphs include the previously determined behavior of the T cell phenotypes (in solid line), as well as a test dose (in dashed line) of a TCT product including a composition with a greater quantity of stem-like memory T cells than effector T cells. As shown in FIG. 7, TCT products including a majority of stem-like memory T cells may be more likely to have greater numbers of total T cells remaining within a patient 200 days post-infusion compared to simulations of TCT products with a majority of effector T cells.

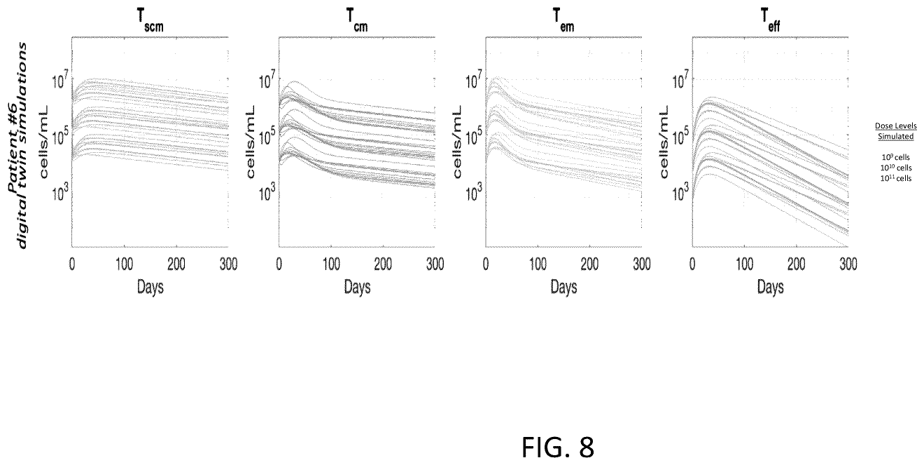

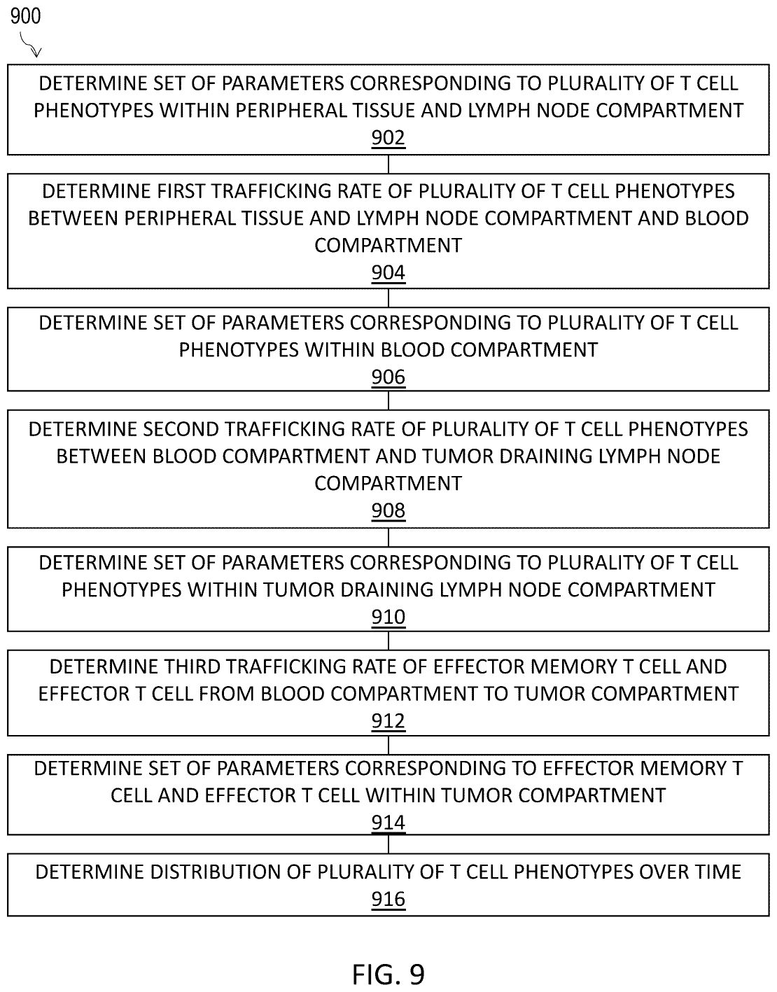

FIG. 8 depicts example graphs showing changes in cellular pharmacokinetics based on phenotypic makeup of a T cell product and dose level, consistent with implementations of the current subject matter. In particular, the graphs in FIG. 8 show a set of simulations performed based on the QSP model 1202. The simulations shown in FIG. 8 included re-simulations of the cellular pharmacokinetics for patient 6, shown in the cellular pharmacokinetics comparisons of FIG. 6A, at three different dose levels (109 cells, 1010 cells, and 1011 cells) and based on a different composition of the TCT product. FIG. 9 depicts a flowchart illustrating an example of a process 900 for determining a distribution of a plurality of T cell phenotypes over time, after delivery of a TCT product, consistent with implementations of the current subject matter. Referring to FIG. 9, the process 900 may be performed by the analysis engine 110 to generate a treatment plan, determine a T cell therapy, including a dose of the TCT product and/or a composition of the TCT product, for treating a tumor, predict a patient response to a dose of a composition of the TCT product, and/or the like. For example, the analysis engine 110 may implement the QSP model 1202 to determine a patient profile including a distribution of T cell phenotypes of the TCT product within physiological compartments of a patient over time, such as for a dose and/or a composition of the TCT product. The T cell therapy, the treatment plan and/or the like, may be determined based at least on the determined distribution. The analysis engine 110 may, based on the architecture 200, capture cellular kinetics of multiple T cell phenotypes following administration of T cell therapies for solid tumors, or the like. Consistent with implementations of the current subject matter, the process 900 refers to the architecture 200 shown in FIG. 2.

At 902, the analysis engine 110 (e.g., at least one data processor) may determine a set of cellular kinetics parameters corresponding to a plurality of T cell phenotypes within a peripheral tissue and lymph node compartment (i.e., healthy tissue compartment) (e.g., the peripheral tissue and lymph node compartment (i.e., healthy tissue compartment) 202) of a patient. The plurality of T cell phenotypes may include a stem-like memory T cell (e.g., the stem-like memory T cell 210), a central memory T cell (e.g., the central memory T cell 212), an effector memory T cell (e.g., the effector memory T cell 214), an effector T cell (e.g., the effector T cell 216), and an endogenous T cell. In some implementations, the plurality of T cell phenotypes include the stem-like memory T cell 210, the central memory T cell 212, the effector memory T cell 214, and the effector T cell 216. The TCT product may include a dose of a composition of the plurality of T cell phenotypes. For example, the dose may include a total quantity of T cells of the TCT product. The composition of the TCT product may include a quantity of each T cell phenotype of the plurality of T cell phenotypes. In other words, the composition of the TCT product may include a particular proportion (or range of proportions) of each T cell phenotype of the plurality of phenotypes forming the TCT product.

The set of cellular kinetics parameters includes at least one of a quantity, a proliferation rate, an apoptosis rate, and a differentiation rate of the plurality of T cell phenotypes. The set of cellular kinetics parameters corresponding to the plurality of T cell phenotypes within the peripheral tissue and lymph node compartment (i.e., healthy tissue compartment) 202 may be determined at a plurality of time points (e.g., a first time point, a second time point, a third time point, and so on). The values of the cellular kinetics parameters of the set of cellular kinetics parameters may be different for each T cell phenotype of the plurality of T cell phenotypes within the peripheral tissue and lymph node compartment (i.e., healthy tissue compartment) 202. Accordingly, the analysis engine 110 may track a behavior of the plurality of T cell phenotypes within the peripheral tissue and lymph node compartment (i.e., healthy tissue compartment) 202 at various time points.

At 904, the analysis engine 110 (e.g., at least one data processor) may determine a first trafficking rate of the plurality of T cell phenotypes between the peripheral tissue and lymph node compartment (i.e., healthy tissue compartment) 202 and a blood compartment (e.g., the blood compartment 204) of the patient. The first trafficking rate may correspond to trafficking of the plurality of T cell phenotypes at lines 220, 224, 228, 232, shown in the architecture 200 of FIG. 2.

At 906, the analysis engine 110 (e.g., at least one data processor) may determine the set of cellular kinetics parameters corresponding to the plurality of T cell phenotypes within the blood compartment 204. The set of cellular kinetics parameters includes at least one of a quantity, a proliferation rate, an apoptosis rate, and a differentiation rate of the plurality of T cell phenotypes within the blood compartment 204. The set of cellular kinetics parameters corresponding to the plurality of T cell phenotypes within the blood compartment 204 may be determined at a plurality of time points (e.g., a first time point, a second time point, a third time point, and so on). The values of the cellular kinetics parameters of the set of cellular kinetics parameters may be different for each T cell phenotype of the plurality of T cell phenotypes within the blood compartment 204. Accordingly, the analysis engine 110 may track a behavior of the plurality of T cell phenotypes within the blood compartment 204 at various time points.

At 908, the platform 120 (e.g., at least one data processor) may determine a second trafficking rate of the plurality of T cell phenotypes between the blood compartment 204 and a tumor draining lymph node compartment (e.g., the tumor draining lymph node compartment 208) of the patient. The second trafficking rate may correspond to trafficking of the plurality of T cell phenotypes at lines 222, 226, 230, 234, shown in the architecture 200 of FIG. 2.

At 910, the analysis engine 110 (e.g., at least one data processor) may determine the set of cellular kinetics parameters corresponding to the plurality of T cell phenotypes within the tumor draining lymph node compartment 208. The set of cellular kinetics parameters includes at least one of a quantity, a proliferation, an apoptosis, and a differentiation of the plurality of T cell phenotypes within the tumor draining lymph node compartment 208. The values of the cellular kinetics parameters of the set of cellular kinetics parameters may be different for each T cell phenotype of the plurality of T cell phenotypes within the tumor draining lymph node compartment 208. In some implementations, the analysis engine 110 determines a differentiation rate parameter (e.g., of the set of cellular kinetics parameters) corresponding to a differentiation of the stem-like memory T cell 210 into the central memory T cell 212, the central memory T cell 212 into the effector memory T cell 214, and the effector memory T cell 214 into the effector T cell 216 within the tumor draining lymph node compartment 208. The set of cellular kinetics parameters corresponding to the plurality of T cell phenotypes within the tumor draining lymph node compartment 208 may be determined at a plurality of time points (e.g., a first time point, a second time point, a third time point, and so on). Accordingly, the analysis engine 110 may track a behavior of the plurality of T cell phenotypes within the tumor draining lymph node compartment 208 at various time points.

At 912, the analysis engine 110 (e.g., at least one data processor) may determine a third trafficking rate of the effector memory T cell 214 and the effector T cell 216 from the blood compartment to a tumor compartment (e.g., the tumor compartment 206) of the patient. The third trafficking rate may correspond to trafficking of the plurality of T cell phenotypes at lines 236, 238, shown in the architecture 200 of FIG. 2.

At 914, the analysis engine 110 (e.g., at least one data processor) may determine the set of cellular kinetics parameters corresponding to the effector memory T cell 214 and the effector T cell 216 within the tumor compartment 206. The values of the cellular kinetics parameters of the set of cellular kinetics parameters may be different for each T cell phenotype of the plurality of T cell phenotypes within the tumor compartment 206. The set of cellular kinetics parameters includes at least one of a quantity, a proliferation rate, an apoptosis rate, and a differentiation rate of the effector memory T cell 214 and the effector T cell 216 within the tumor compartment 206. In some implementations, the analysis engine 110 determines a differentiation rate parameter (e.g., of the set of cellular kinetics parameters) corresponding to a differentiation rate of the effector memory T cell 214 into the effector T cell 216 within the tumor compartment 206. The set of cellular kinetics parameters corresponding to the effector memory T cell 214 and the effector T cell 216 within the tumor compartment 206 may be determined at a plurality of time points (e.g., a first time point, a second time point, a third time point, and so on). Accordingly, the analysis engine 110 may track a behavior of the effector memory T cell 214 and the effector T cell 216 within the tumor compartment 206 at various time points.

At 916, the analysis engine 110 (e.g., at least one data processor) may determine a distribution of each of the plurality of T cell phenotypes in the peripheral tissue and lymph node compartment (i.e., healthy tissue compartment) 202, the blood compartment 204, the tumor draining lymph node compartment 208, and the tumor compartment 206 over time, based at least on the set of cellular kinetics parameters, the first trafficking rate, the second trafficking rate, and the third trafficking rate. In some implementations, the analysis engine 110 may determine a persistence of the plurality of T cell phenotypes in the peripheral tissue and lymph node compartment (i.e., healthy tissue compartment) 202, the blood compartment 204, the tumor draining lymph node compartment 208, and the tumor compartment 206 over time, such as based at least on the set of cellular kinetics parameters, the first trafficking rate, the second trafficking rate, and the third trafficking rate, and/or based on the determined distribution.

The distribution may include a first distribution corresponding to a dose of a composition of the TCT product administered to a first patient and a second distribution corresponding to a dose of a composition of the TCT product administered to a second patient. Based at least on the first distribution corresponding to the first patient and/or the second distribution corresponding to the second patient (among other distributions and patients), the analysis engine 110 may determine a response of another (e.g., a third) patient to a T cell therapy including a dose and a composition of the TCT product. Based at least on the determined response of the third patient to the T cell therapy, the analysis engine 110 may determine a treatment plan for the third patient to treat a tumor and/or cancer of the third patient. In other words, the treatment plan may be determined based at least on the predicted distribution (e.g., the first distribution and/or the second distribution) corresponding to a particular dose and/or composition of the TCT product. The T cell therapy may be administered to the patient according to the treatment plan including the determined dose and composition of the TCT product. In some implementations, the dose of the composition of the TCT product is administered to the first patient after administration of a lymphodepletion regimen to the first patient and the dose of the composition of the TCT product is administered to the second patient after administration of a lymphodepletion regimen to the second patient. The lymphodepletion regimens administered to each patient may be the same or different. Accordingly, in some implementations, the analysis engine 110 determines a T cell therapy for treating a tumor including a dose of a composition of the plurality of T cell phenotypes of the TCT product. For example, in some implementations, the T cell therapy may be determined based at least on the predicted distribution (e.g., the first distribution and/or the second distribution) corresponding to a particular dose and/or composition of the TCT product. As described herein, the T cell therapy may include at least one of a T cell receptor (TCR)-engineered T cell therapy, an autologous T cell therapy, an allogeneic T cell therapy, an iPSC-derived T cell therapy, and a CAR T cell therapy.

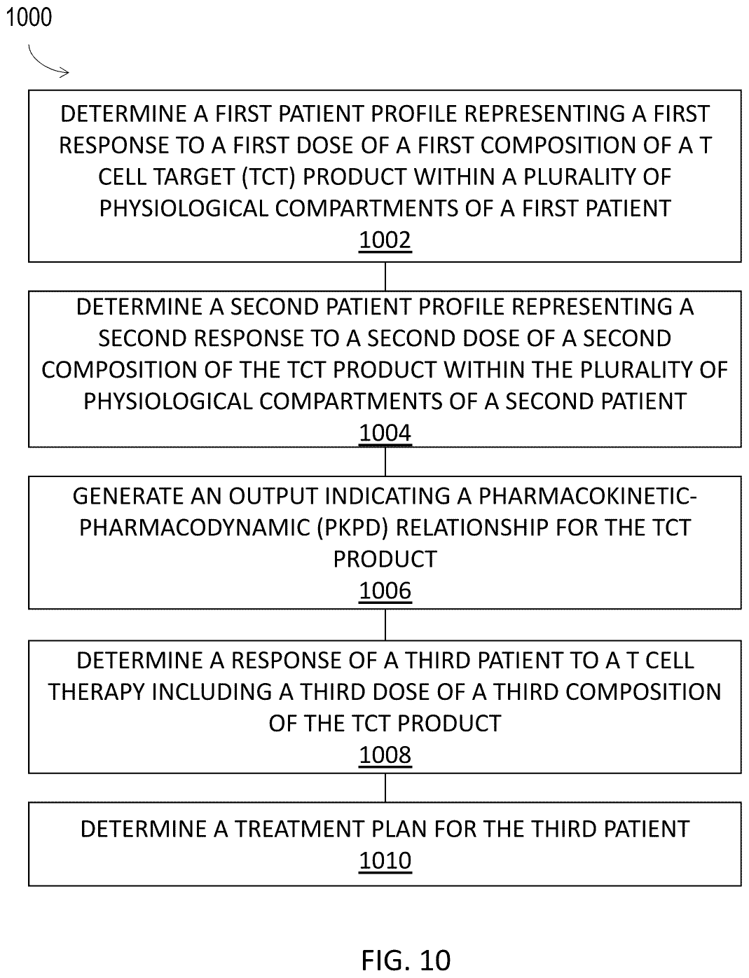

FIG. 10 depicts a flowchart illustrating an example of a process 1000 for determining a treatment plan for a patient, consistent with implementations of the current subject matter. Referring to FIG. 10, the process 1000 may be performed by the analysis engine 110 to generate the treatment plan, determine a T cell therapy including a dose of the TCT product and/or a composition of the TCT product for treating a tumor, predict a patient response to a dose of a composition of the TCT product, and/or the like. For example, the analysis engine 110 may implement the QSP model 1202 to determine a patient profile including a distribution of T cell phenotypes of the TCT product within physiological compartments of a patient over time, such as for a dose and/or a composition of the TCT product. The T cell therapy, the treatment plan and/or the like, may be determined based at least on the determined distribution. Consistent with implementations of the current subject matter, the process 1000 refers to the architecture 200 shown in FIG. 2. Further, consistent with implementations of the current subject matter, the process 1000 may include one or more steps of the process 900, and the process 900 may include one or more steps of the process 1000.

At 1002, the analysis engine 110 (e.g., at least one data processor) may determine a first patient profile representing a first response to a first dose of a first composition of a T cell target (TCT) product within a plurality of physiological compartments of a first patient. The first patient profile includes a first set of cellular kinetics parameters including at least one of a first quantity, a first proliferation rate, a first trafficking rate a first apoptosis rate, and a first differentiation rate of a plurality of T cell phenotypes within at least one of the plurality of physiological compartments of the first patient over a period of time. Thus, the first patient profile may include one or more cellular kinetics parameters (e.g., the first set of cellular kinetics parameters) of the set of cellular kinetics parameters, as described herein. Further, consistent with implementations of the current subject matter, the plurality of physiological compartments includes a peripheral tissue and lymph node compartment (i.e., healthy tissue compartment) 202, a blood compartment 204, a tumor draining lymph node compartment 208, and a tumor compartment 206.

The plurality of T cell phenotypes includes at least two of a stem-like memory T cell 210, a central memory T cell 212, an effector memory T cell 214, an effector T cell 216, and an endogenous T cell. The first composition of the TCT product may include a first quantity of each of the plurality of T cell phenotypes. The first patient profile includes a first response corresponding to each of the plurality of T cell phenotypes.

At 1004, the analysis engine 110 (e.g., at least one data processor) may determine a second patient profile representing a second response to a second dose of a second composition of a T cell target (TCT) product within a plurality of physiological compartments of a second patient. The second patient profile includes a second set of cellular kinetics parameters including at least one of a second quantity, a second proliferation rate, a second trafficking rate, a second apoptosis rate, and a second differentiation rate of a plurality of T cell phenotypes within at least one of the plurality of physiological compartments of the second patient over a period of time. Thus, the second patient profile may include one or more cellular kinetics parameters (e.g., the second set of cellular kinetics parameters) of the set of cellular kinetics parameters, as described herein.

The plurality of T cell phenotypes includes at least two of a stem-like memory T cell 210, a central memory T cell 212, an effector memory T cell 214, an effector T cell 216, and an endogenous T cell. The second composition of the TCT product may include a second quantity of each of a plurality of T cell phenotypes. The second patient profile includes a second response corresponding to each of the plurality of T cell phenotypes.

In some implementations, the first composition is different from the second composition and the first dose is different from the second dose. A linear increase from the first dose to the second dose can result in a nonlinear change between the first response and the second response.

At 1006, the analysis engine 110 (e.g., at least one data processor) generates an output indicating a pharmacokinetic-pharmacodynamic (PKPD) relationship for the TCT product based at least on the first patient profile and the second patient profile. The PKPD relationship may be schematically illustrated as the architecture 200 of the QSP model 1202. The PKPD relationship may at least represent a distribution of each of the plurality of T cell phenotypes in the physiological compartments of the patient over time.

At 1008, the analysis engine 110 (e.g., at least one data processor) determines a response to a third patient to a T cell therapy including a third dose of a third composition of the TCT product. As described herein, the T cell therapy is at least one of a T cell receptor (TCR)-engineered T cell therapy, an autologous T cell therapy, an allogeneic T cell therapy, an iPSC-derived T cell therapy, and a CAR T cell therapy. In some implementations, determining the response in the third patient to the T cell therapy includes simulating, based at least on the output, a plurality of responses to a plurality of doses of a plurality of compositions of the TCT product in a plurality of simulated patients. In some implementations, simulating the plurality of responses is based on application of a lymphodepletion regimen to the plurality of simulated patients. The response in the third patient may be one of the plurality of simulated responses. This allows for determination of an ideal dose and/or composition of the TCT product for treating a tumor and/or cancer in the third patient. Further, the third patient may be determined by at least varying at least one of the first dose, the first composition, the second dose, and the second composition. Such configurations may also allow for determination of an ideal dose and/or composition of the TCT product for treating a tumor and/or cancer in the third patient.

At 1010, the analysis engine 110 (e.g., at least one data processor) determines a treatment plan for the third patient, based at least on the response of the third patient to the T cell therapy including the third dose of the third composition of the TCT product. In other words, the treatment plan may be determined based at least on the predicted PKPD relationship, the first patient profile, the second patient profile, the predicted response of the third patient, and/or the like. The T cell therapy may be administered to the third patient according to the treatment plan.

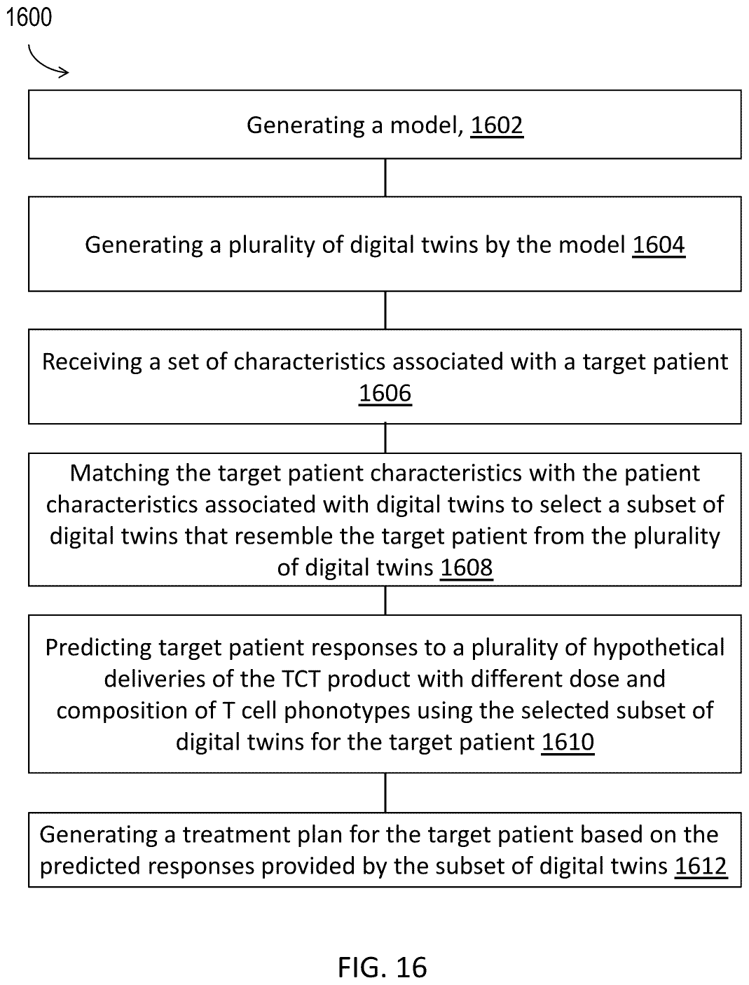

FIG. 16 depicts a flowchart illustrating an example of a process 1600 for generating personalized treatment plans using the model and the digital twins, consistent with implementations of the current subject matter. Referring to FIG. 16, the process 1600 may be performed by the platform 120 to generate a treatment plan, determine a T cell therapy, including a dose of the TCT product and/or a composition of the TCT product, for treating a tumor, predict a patient response to a dose of a composition of the TCT product, and/or the like. For example, the platform 120 may, at operation 1602, generate a model, wherein the model may be a QSP model, such as the QSP model generated by the model generation engine 122 using the architecture 200 shown in FIG. 2. In some embodiments, the generated model may simulate a distribution of each of a plurality of T cell phenotypes in a plurality of physiological compartments over time after delivery a T cell target (TCT) product to a patient. In some embodiments, the process 1600 may proceed to operation 1604, wherein the digital twins generation engine 124 may generate a plurality of digital twins by the model, for example, by the QSP model 1202. In some embodiments, each of the plurality of digital twins may represent a distribution of the plurality of T cell phenotypes in the plurality of physiological compartments over time associated with a corresponding patient, wherein the corresponding patient has a set of patient characteristics. In some embodiment, the patient characteristics may comprise patient biometrics, patient medical history, and baseline biomarker data. In some embodiment, the patient characteristics may comprise the type of cancer/tumor the corresponding patient has. Next, the process 1600 may proceed to operation 1606, wherein the matching engine 126 may receive a set of characteristics associated with a target patient. In some embodiments, the set of characteristics may be received from a client node 102 or 106 as shown in FIG. 1. Next, the matching engine 126 may match, at operation 1608, the target patient characteristics with the patient characteristics associated with digital twins to select a subset of digital twins that resemble the target patient from the plurality of digital twins. In some embodiments, the matching engine 126 may compute matching scores for each digital twin within the collection concerning the target patient. Subsequently, the matching engine 126 may select the top N digital twins with the highest scores. This may facilitate a selection of a subset of digital twins from the collection of digital twins that resemble the target patient among the available digital twins. Next, the process 1600 may proceed to operation 1610, wherein the platform 120 may predict target patient responses to a plurality of hypothetical deliveries of the TCT product with different dose and composition of T cell phonotypes using the selected subset of digital twins for the target patient. The treatment plan generation engine 128 may then, at operation 1612, generate a treatment plan for the target patient based on the predicted responses provided by the subset of digital twins. In some embodiments, the treatment plan may be predicted to provide a T cell persistence level above a predetermined threshold over time in the plurality of physiological compartments of the target patient, wherein the treatment plan provides/comprises dose and composition of the plurality of T cell phonotypes of the treatment. In some embodiments, the treatment plan may be predicted to provide a T cell persistence level above a predetermined threshold over time in at least one of the plurality of physiological compartments of the target patient. In some embodiments, the treatment plan may be predicted to provide a T cell persistence level above a predetermined threshold over time in at least the tumor compartment of the target patient.

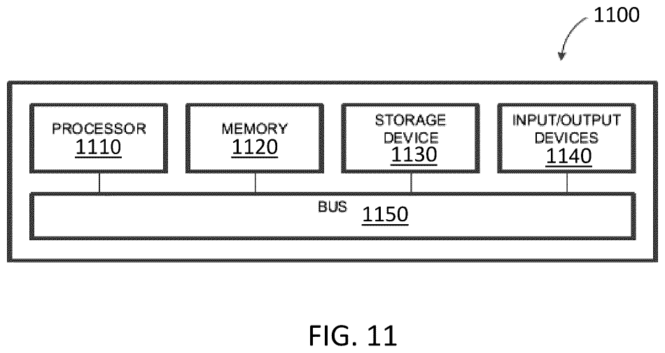

FIG. 11 depicts a block diagram illustrating a computing system 1100 consistent with implementations of the current subject matter. Referring to FIGS. 1-10, the computing system 1100 can be used to implement the analysis engine 110, the QSP model 1202, and/or any components therein.

As shown in FIG. 11, the computing system 1100 can include a processor 1110, a memory 1120, a storage device 1130, and input/output devices 1140. The processor 1110, the memory 1120, the storage device 1130, and the input/output devices 1140 can be interconnected via a system bus 1150. The computing system 1100 may additionally or alternatively include a graphic processing unit (GPU), such as for image processing, and/or an associated memory for the GPU. The GPU and/or the associated memory for the GPU may be interconnected via the system bus 1150 with the processor 1110, the memory 1120, the storage device 1130, and the input/output devices 1140. The memory associated with the GPU may store one or more images described herein, and the GPU may process one or more of the images described herein. The GPU may be coupled to and/or form a part of the processor 1110. The processor 1110 is capable of processing instructions for execution within the computing system 1100. Such executed instructions can implement one or more components of, for example, the analysis engine 110, the QSP model 1202, and/or the like. In some implementations of the current subject matter, the processor 1110 can be a single-threaded processor. Alternately, the processor 1110 can be a multi-threaded processor. The processor 1110 is capable of processing instructions stored in the memory 1120 and/or on the storage device 1130 to display graphical information for a user interface provided via the input/output device 1140.

The memory 1120 is a computer readable medium such as volatile or non-volatile that stores information within the computing system 1100. The memory 1120 can store data structures representing configuration object databases, for example. The storage device 1130 is capable of providing persistent storage for the computing system 1100. The storage device 1130 can be a floppy disk device, a hard disk device, an optical disk device, or a tape device, or other suitable persistent storage means. The input/output device 1140 provides input/output operations for the computing system 1100. In some implementations of the current subject matter, the input/output device 1140 includes a keyboard and/or pointing device. In various implementations, the input/output device 1140 includes a display unit for displaying graphical user interfaces.

According to some implementations of the current subject matter, the input/output device 1140 can provide input/output operations for a network device. For example, the input/output device 1140 can include Ethernet ports or other networking ports to communicate with one or more wired and/or wireless networks (e.g., a local area network (LAN), a wide area network (WAN), the Internet).

In some implementations of the current subject matter, the computing system 1100 can be used to execute various interactive computer software applications that can be used for organization, analysis and/or storage of data in various (e.g., tabular) format (e.g., Microsoft Excel®, and/or any other type of software). Alternatively, the computing system 1100 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1140. The user interface can be generated and presented to a user by the computing system 1100 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

What is claimed is:

1. A method, comprising:
generating a model, wherein the model simulates a distribution of each of a plurality of T cell phenotypes in a plurality of physiological compartments over time after delivery a T cell target (TCT) product to a patient;
generating a plurality of digital twins by the model, wherein each of the plurality of digital twins represents a distribution of the plurality of T cell phenotypes in the plurality of physiological compartments over time associated with a corresponding patient, wherein the corresponding patient has a set of patient characteristics;
receiving a set of characteristics associated with a target patient;
matching the target patient characteristics with the patient characteristics associated with digital twins to select a subset of digital twins that resemble the target patient from the plurality of digital twins;
predicting target patient responses to a plurality of hypothetical deliveries of the TCT product with different dose and composition of T cell phonotypes using the selected subset of digital twins for the target patient, and
generating a treatment plan for the target patient based on the predicted responses provided by the subset of digital twins, wherein the treatment plan is predicted to provide a T cell persistence level above a predetermined threshold over time in at least one of the plurality of physiological compartments of the target patient, wherein the treatment plan comprises dose and composition of the plurality of T cell phonotypes of the treatment.

2. The method of claim 1, further comprising:
identifying sources of variability across patients by visualizing a parameter space for a second subset of the plurality of digital twins; and
modifying the model to account for the sources of variability across patients.

3. The method of claim 1, further comprising:
generating a plurality of simulations across different T cell phonotypes composition and dose level by the model;
visualizing a distribution of each of the plurality of T cell phenotypes over time associated with the plurality of simulations, wherein the visualization is indicative of an impact of T cell phonotypes composition on T cell persistence level over time.

4. The method of claim 1, wherein the set of patient characteristics comprises patient biometrics, patient medical history, and baseline biomarker data.

5. The method of claim 1, wherein each of the plurality of digital twins further represents a response to a dose of a composition of the TCT product within the plurality of compartments.

6. The method of claim 5, wherein each of the plurality of digital twins comprises a set of cellular kinetics parameters including at least one of a quantity, a proliferation rate, a trafficking rate, an apoptosis rate, and a differentiation rate of the plurality of T cell phenotypes within at least one of the plurality of physiological compartments of the corresponding patient over a period of time.

7. The method of claim 6, wherein the composition of TCT product comprises an initial quantity of each of the plurality of T cell phenotypes.

8. The method of claim 1, wherein the plurality of T cell phenotypes comprises at least two of a stem-like memory T cell, a central memory T cell, an effector memory T cell, an effector T cell, and an endogenous T cell.

9. The method of claim 1, wherein the plurality of physiological compartments includes a peripheral tissue and lymph node compartment, a blood compartment, a tumor draining lymph node compartment, and a tumor compartment.

10. The method of claim 1, wherein generating the model further comprises:
determining, by at least one data processor, a set of cellular kinetics parameters corresponding to the plurality of T cell phenotypes within a peripheral tissue and lymph node compartment of a patient after delivery of a T cell target (TCT) product, wherein the plurality of T cell phenotypes includes a stem-like memory T cell, a central memory T cell, an effector memory T cell, an effector T cell, and an endogenous T cell;
determining, by the at least one data processor, a first trafficking rate of the plurality of T cell phenotypes between the peripheral tissue and lymph node compartment and a blood compartment of the patient;
determining, by the at least one data processor, the set of cellular kinetics parameters corresponding to the plurality of T cell phenotypes within the blood compartment;
determining, by the at least one data processor, a second trafficking rate of the plurality of T cell phenotypes between the blood compartment and a tumor draining lymph node compartment of the patient;
determining, by the at least one data processor, the set of cellular kinetics parameters corresponding to the plurality of T cell phenotypes within the tumor draining lymph node compartment;
determining, by the at least one data processor, a third trafficking rate of the effector memory T cell and the effector T cell from the blood compartment to a tumor compartment of the patient;
determining, by the at least one data processor, the set of cellular kinetics parameters corresponding to the effector memory T cell and the effector T cell within the tumor compartment; and
determining, by the at least one data processor and based on the set of cellular kinetics parameters, the first trafficking rate, the second trafficking rate, and the third trafficking rate, a distribution of each of the plurality of T cell phenotypes in the peripheral tissue and lymph node compartment, the blood compartment, the tumor draining lymph node compartment, and the tumor compartment over time.

11. The method of claim 10, further comprising: determining, by the at least one data processor, a differentiation rate parameter corresponding to a differentiation of the effector memory T cell into the effector T cell within the tumor compartment; and wherein determining the distribution is further based on the differentiation rate parameter.

12. The method of claim 10, further comprising: determining, by the at least one data processor, a differentiation rate parameter corresponding to a differentiation of the stem-like memory T cell into the central memory T cell, the central memory T cell into the effector memory T cell, and the effector memory T cell into the effector T cell within the tumor draining lymph node compartment; and wherein determining the distribution is further based on the differentiation rate parameter.

13. The method of claim 10, further comprising:
determining a T cell therapy for treating a tumor,
wherein the T cell therapy includes a dose of a composition of the plurality of T cell phenotypes of the TCT product, and wherein the T cell therapy is at least one of a T cell receptor (TCR)-engineered T cell therapy, an autologous T cell therapy, an allogeneic T cell therapy, an iPSC-derived T cell therapy, and a CAR T cell therapy.

14. A method, comprising:
determining, by at least one data processor, a first patient profile representing a first response to a first dose of a first composition of a T cell target (TCT) product within a plurality of physiological compartments of a first patient;
determining, by the at least one data processor, a second patient profile representing a second response to a second dose of a second composition of the TCT product within the plurality of physiological compartments of a second patient;
generating, by the at least one data processor and based at least on the first patient profile and the second patient profile, an output indicating a pharmacokinetic-pharmacodynamic (PKPD) relationship for the TCT product;
determining, by the at least one data processor and based at least on the output, a response of a third patient to a T cell therapy including a third dose of a third composition of the TCT product; and
determining, based at least on the response of the third patient to the T cell therapy including the third dose of the third composition of the TCT product, a treatment plan for the third patient.

15. The method of claim 14, wherein the first patient profile includes a first set of cellular kinetics parameters including at least one of a first quantity, a first proliferation rate, a first trafficking rate, a first apoptosis rate, and a first differentiation rate of a plurality of T cell phenotypes within at least one of the plurality of physiological compartments of the first patient over a period of time; and
wherein the second patient profile includes a second set of cellular kinetics parameters including at least one of a second quantity, a second proliferation rate, a second trafficking rate, a second apoptosis rate, and a second differentiation rate of the plurality of T cell phenotypes within at least one of the plurality of physiological compartments of the second patient over the period of time.

16. The method of claim 15, wherein the first composition includes a first quantity of each of a plurality of T cell phenotypes; and wherein the second composition includes a second quantity of each of the plurality of T cell phenotypes.

17. The method of claim 16, wherein the first patient profile and the second patient profile each include responses corresponding to each of the plurality of T cell phenotypes.

18. The method of claim 17, wherein the plurality of T cell phenotypes includes at least two of a stem-like memory T cell, a central memory T cell, an effector memory T cell, an effector T cell, and an endogenous T cell.

19. The method of claim 14, wherein the plurality of physiological compartments includes a peripheral tissue and lymph node compartment, a blood compartment, a tumor draining lymph node compartment, and a tumor compartment.

20. The method of claim 14, wherein the T cell therapy is at least one of a T cell receptor (TCR)-engineered T cell therapy, an autologous T cell therapy, an allogeneic T cell therapy, an iPSC-derived T cell therapy, and a CAR T cell therapy.

21. The method of claim 14, wherein a linear increase from the first dose to the second dose results in a nonlinear change between the first response and the second response.

22. The method of claim 14, wherein the first composition is different from the second composition; and wherein the first dose is different from the second dose.

23. The method of claim 14, wherein determining the response in the third patient to the T cell therapy includes: simulating, based at least on the output, a plurality of responses to a plurality of doses of a plurality of compositions of the TCT product in a plurality of simulated patients; and wherein the response in the third patient is one of the plurality of simulated responses.

24. The method of claim 14, wherein the first patient profile is determined after administration of a first lymphodepletion regimen to the first patient; and wherein the second patient profile is determined after administration of a second lymphodepletion regimen to the second patient.

25. The method of claim 14, wherein the response in the third patient is determined by at least varying at least one of the first dose, the first composition, the second dose, and the second composition.

26. The method of claim 23, wherein simulating the plurality of responses is based on application of a lymphodepletion regimen to the plurality of simulated patients.

27. A system comprising: one or more processors; and a non-transitory memory coupled to the processors comprising instructions executable by the processors, the processors operable when executing the instructions to:
determine a first patient profile representing a first response to a first dose of a first composition of a T cell target (TCT) product within a plurality of physiological compartments of a first patient;
determine a second patient profile representing a second response to a second dose of a second composition of the TCT product within the plurality of physiological compartments of a second patient;
generate, based at least on the first patient profile and the second patient profile, an output indicating a pharmacokinetic-pharmacodynamic (PKPD) relationship for the TCT product;
determine, based at least on the output, a response of a third patient to a T cell therapy including a third dose of a third composition of the TCT product; and
determine, based at least on the response of the third patient to the T cell therapy including the third dose of the third composition of the TCT product, a treatment plan for the third patient.

28. One or more computer-readable non-transitory storage media embodying software that is operable when executed to:
determine a first patient profile representing a first response to a first dose of a first composition of a T cell target (TCT) product within a plurality of physiological compartments of a first patient;
determine a second patient profile representing a second response to a second dose of a second composition of the TCT product within the plurality of physiological compartments of a second patient;
generate, based at least on the first patient profile and the second patient profile, an output indicating a pharmacokinetic-pharmacodynamic (PKPD) relationship for the TCT product;
determine, based at least on the output, a response of a third patient to a T cell therapy including a third dose of a third composition of the TCT product; and
determine, based at least on the response of the third patient to the T cell therapy including the third dose of the third composition of the TCT product, a treatment plan for the third patient.

29. A method, comprising:
maintaining, at a database, a plurality of patient profiles, wherein the plurality of patient profiles are indicative of patient responses to a dose of a composition of a T cell target (TCT) product in a format of a distribution of a plurality of T cell phenotypes in a plurality of physiological compartments over time,
receiving, by at least one data processor, a baseline biomarker dataset associated with a target patient;
determining, by the at least one data processor, a set of cellular kinetics parameters based on the baseline biomarker dataset for the target patient;
generating, by the at least one data processor, a plurality of simulations associated with the target patient based at least in part on the cellular kinetics parameters;
selecting, by the at least one data processor, a subset of simulations from the plurality of simulations based on a similarity level between the target patient and a patient, and
generating a treatment plan for the target patient based on a set of predicted responses generated by the subset of simulations.
